SEQUENCE LISTING (13 sequences)

SEQ ID NO: 1 - protein, 18 aa, synthetic construct
ECGLLPVGRPDRVWRLCK

SEQ ID NO: 2 - DNA, 20 bp, synthetic construct
  source 1..20: mol_type = other DNA
ctaaacagatgaagtgctcc

SEQ ID NO: 3 - DNA, 18 bp, synthetic construct
  source 1..18: mol_type = other DNA
ggtcattctcctggaagg

SEQ ID NO: 4 - DNA, 20 bp, synthetic construct
  source 1..20: mol_type = other DNA
gcagaaaaaggcaaagaatc

SEQ ID NO: 5 - DNA, 18 bp, synthetic construct
  source 1..18: mol_type = other DNA
ctacatttgccgaagagc

SEQ ID NO: 6 - DNA, 21 bp, synthetic construct
  source 1..21: mol_type = other DNA
gggcagggttatttaatatgg

SEQ ID NO: 7 - DNA, 18 bp, synthetic construct
  source 1..18: mol_type = other DNA
aatcaggcatcgagacag

SEQ ID NO: 8 - DNA, 20 bp, synthetic construct
  source 1..20: mol_type = other DNA
aatgtgaatgcagaccaaag

SEQ ID NO: 9 - DNA, 21 bp, synthetic construct
  source 1..21: mol_type = other DNA
gacttataccgggatttcttg

SEQ ID NO: 10 - DNA, 22 bp, synthetic construct
  source 1..22: mol_type = other DNA
caaggacctacgagaaaattac

SEQ ID NO: 11 - DNA, 20 bp, synthetic construct
  source 1..20: mol_type = other DNA
atcacagtttggaatttggg

SEQ ID NO: 12 - DNA, 20 bp, synthetic construct
  source 1..20: mol_type = other DNA
cccagaaggaagtacatttg

SEQ ID NO: 13 - DNA, 20 bp, synthetic construct
  source 1..20: mol_type = other DNA
gtttaacaggtaactcgtgc

(19) United States
(12) Patent Application Publication
(10) Pub. No.: US 2024/0181125 A1
(43) Pub. Date: Jun. 6, 2024

(54) COMPOSITIONS AND METHODS FOR SCARLESS WOUND HEALING IN DIABETES

(71) Applicant: Washington University, St. Louis, MO (US)

(72) Inventors: Jianjun Guan, St. Louis, MO (US); Hong Niu, St. Louis, MO (US); Ya Guan, St. Louis, MO (US)

(73) Assignee: Washington University, St. Louis, MO (US)

(21) Appl. No.: 18/516,382

(22) Filed: Nov. 21, 2023

Related U.S. Application Data
(60) Provisional application No. 63/384,813, filed on Nov. 23, 2022.

(51) Int. Cl. A61L 26/00; C08L 33/26

(52) U.S. Cl. CPC A61L 26/008; A61L 26/0014; A61L 26/0066; C08L 33/26; A61L 2300/252

(57) ABSTRACT

A method of promoting healing of a wound in a diabetic patient is disclosed that includes applying a hydrogel loaded with a therapeutically effective dosage of a TGF receptor II (TGFβRII) inhibitor to the wound.

### CROSS-REFERENCE TO RELATED APPLICATIONS

This application claims priority from U.S. Provisional Application Ser. No. 63/384,813 filed on Nov. 23, 2022, the content of which is incorporated by reference herein in its entirety.

### STATEMENT REGARDING FEDERALLY SPONSORED RESEARCH OR DEVELOPMENT

This invention was made with government support under EB022018 awarded by the National Institutes of Health. The government has certain rights in the invention.

### MATERIAL INCORPORATED-BY-REFERENCE

The Sequence Listing, which is a part of the present disclosure, includes a computer readable form comprising nucleotide and/or amino acid sequences of the present invention (file name “019288-US—NP_SEQ_LISTING.xml” created on 21 Nov. 2023; 12,463 bytes). The subject matter of the Sequence Listing is incorporated herein by reference in its entirety.

### FIELD OF THE DISCLOSURE

The present disclosure generally relates to compositions and methods for promoting scarless wound healing in diabetic patients.

### BACKGROUND OF THE DISCLOSURE

Diabetes affects more than 29 million people in the United States alone. Non-healing chronic wounds are a major complication of diabetes that can lead to amputation or death. While the mechanisms of slow diabetic wound healing are not fully understood, it has been recognized that chronic inflammation, delayed angiogenesis, and abnormal functions (particularly migration) of skin cells are major triggers. Therefore, simultaneous decrease of tissue inflammation, stimulation of angiogenesis, and increase of skin cell migration will efficiently accelerate diabetic wound healing. However, this cannot be achieved by existing approaches. There is a clinical need for more effective treatments for diabetic wounds.

### SUMMARY OF THE DISCLOSURE

In various aspects, a method of promoting healing of a wound in a diabetic patient is disclosed that includes applying a hydrogel loaded with a therapeutically effective dosage of a TGFβ receptor II (TGFβRII) inhibitor to the wound. In some aspects, the TGFβ receptor II (TGFβRII) inhibitor is a peptide comprising the amino acid sequence ECGLLPVGRPDRVWRLCK (SEQ ID NO: 1) and variations thereof. In some aspects, the hydrogel includes a thermoresponsive N-isopropylacrylamide (NIPAAm) hydrogel. In some aspects, the NIPAAm hydrogel includes from about 2 wt % to about 20 wt % of NIPAAm. In some aspects, the NIPAAm hydrogel includes from about 6 wt % to about 10 wt % of NIPAAm. In some aspects, the NIPAAm hydrogel includes about 6 wt % of NIPAAm. In some aspects, the NIPAAm hydrogel includes an injectable liquid phase at 4° C. and a gel phase at 37° C. In some aspects, the therapeutically effective dosage of the TGFβ receptor II (TGFβRII) inhibitor ranges from about 1 μg/mL to about 100 μg/mL. In some aspects, the therapeutically effective dosage of the TGFβ receptor II (TGFβRII) inhibitor ranges from about 1 μg/mL to about 10 μg/mL. In some aspects, the hydrogel is configured to degrade in the presence of reactive oxygen species (ROS). In some aspects, the hydrogel is synthesized by free radical polymerization using N-isopropylacrylamide (NIPAAm), 2-hydroxyethyl methacrylate (HEMA), and 4-(hydroxymethyl)-phenylboronic acid pinacol ester (HPPE).

Other objects and features will be in part apparent and in part pointed out hereinafter.

### DESCRIPTION OF THE DRAWINGS

The patent or application file contains at least one drawing executed in color. Copies of this patent or patent application publication with color drawing(s) will be provided by the Office upon request and payment of the necessary fee.

Those of skill in the art will understand that the drawings, described below, are for illustrative purposes only. The drawings are not intended to limit the scope of the present teachings in any way.

FIG. 1A is a photograph of the NIPAAm hydrogel in a liquid state at 4ºC.

FIG. 1B is a photograph of the NIPAAm hydrogel of FIG. 1A in a liquid state as it is drawn into a surgical needle.

FIG. 1C is a photograph of the NIPAAm hydrogel of FIG. 1A in a solid state at 37° C.

FIG. 1D is a series of graphs summarizing the degradation characteristics of NIPAAm hydrogels with varying water contents in the presence of H₂O₂.

FIG. 2 is a graph summarizing the ECG release characteristics of NIPAAm hydrogels loaded with varying payload concentrations of ECG.

FIG. 3A is a graph summarizing wound healing of mice treated with an ECG-loaded NIPAAm hydrogel.

FIG. 3B contains a series of photographs of dermal wounds over a 2-week period of no treatment (top row), treatment with an unloaded NIPAAm hydrogel (middle row), or treatment with an ECG-loaded NIPAAm hydrogel (bottom row).

FIG. 4A is a Western blot showing in vivo expression of p38 (left) and GAPHD (control, right) after treatment with an unloaded NIPAAm (Gel) or with an ECG-loaded NIPAAm hydrogel (Gel/ECG).

FIG. 4B contains a Western blot (top) showing in vitro expression of dermal fibroblasts under high glucose levels of p38 and GAPHD (control) after treatment with two dosages of ECG in the presence of TGFβ, and a graph (bottom) summarizing the p38 expression shown in the Western blot.

FIG. 5A is a graph of the binding affinity between PTβR2I and TβR1/TβR2/TβR3/IgG, tested by the binding assay (n=8).

FIG. 5B is a graph of the Kd values of PTβR2I in binding with TβR2 as measured by an Elisa-like binding assay (n=8).

FIG. 5C is a graph of the Kd values of PTβR2I in binding with TβR1 as measured by an Elisa-like binding assay (n=8).

FIG. 5D is a graph of the Kd values of PTβR2I in binding with TβR3 as measured by an Elisa-like binding assay (n=8).

FIG. 5E is a graph of the Kd values of PTβR2I in binding with IgG as measured by an Elisa-like binding assay (n=8).

FIG. 5F is a set of images showing PTβR2I binding specificity as tested using immunofluorescence (IF) staining by staining the HDFs with anti-TβR2 antibody. The PTβR2I treated group showed evident expression of FITC, and no unbound TβR2 was observed. Scale bar=50 μm.

FIG. 5G is a set of images showing the competitive binding affinity to TβR2 as examined by using IF staining with anti-TβR2 antibody, with a total treatment time of 48 hours. Scale bar=50 μm.

FIG. 5H is a graph showing the competitive binding between PTβR2I and TGFβ1 as determined using fluorescence intensity measurements by a plate reader (excitation/emission=485/535 nm). The Non-PTβR2I-treated group served as a control. The total time for all treatments was 48 hours (n=10). p>0.05 for any pair of the groups with the addition of PTβR2I.

FIG. 5I is a schematic showing how PTβR2I binds to TβR2 on HDF and how it surpasses TGFβI in binding ability.

FIG. 5J is a graph showing dsDNA of HaCaT treated with TGFβ1 and PTβR2I at day 3 (n=4).

FIG. 5K is a graph showing dsDNA of dermal fibroblasts at day 3 (n=5).

FIG. 5L is a graph showing the dsDNA of endothelial cells at day 3 (n=6).

FIG. 5M is a set of representative images and quantification of migration using a scratch assay for HaCaT at 0 and 48 hours (n=4).

FIG. 5N is a set of representative images and quantification of migration for dermal fibroblasts at 0 and 72 hours (n=7).

FIG. 5O is a set of representative images and quantification of migration for endothelial cells at 0 and 36 hours (n=7).

FIG. 5P is a set of representative images of endothelial cell lumen formation 24 hours posttreatment. The cytoskeleton was stained by F-actin.

FIG. 5Q is a graph of the quantification of lumen density based on the images (n=6).

FIG. 5R is a graph of fold change for PDGFBB, VEGFA, and HGF in endothelial cells.

FIG. 5S is a graph of fold change for IL6, TNFA, PDGFBB, and VEGFA expressed from macrophages.

FIG. 5T is a graph of fold change for IL1B, IL6, TNFA, PDGFBB, and IGF1 in dermal fibroblasts.

FIG. 5U is a graph of fold change for IL1B, IL6, TNFA, PDGFBB, and HGF in HaCaT treated with no TGFβ1, TGFβ1, or PTβR2I and TGFβ1.

FIG. 5V is an immunoblot of p-p38, p-Smad2/3, and α-SMA derived from dermal fibroblasts. GAPDH was used as a loading control (n=3). The cells were treated with no TGFβ1, TGFβ1, and PTβR2I with TGFβ1 under high glucose. All data demonstrated as mean±standard deviation. Data were analyzed by one-way ANOVA with Bonferroni post-test (n.s.p>0.05, *p<0.001).

FIG. 5W is graph quantifying an immunoblot of p-p38, p-Smad2/3, and α-SMA derived from dermal fibroblasts. GAPDH was used as a loading control (n=3). The cells were treated with no TGFβ1, TGFβ1, and PTβR2I with TGFβ1 under high glucose. All data demonstrated as mean±standard deviation. Data were analyzed by one-way ANOVA with Bonferroni post-test (n.s.p>0.05,*p<0.001).

FIG. 6A is a chemical structure of polymer (NIPAAm-co-HEMA-co-AHPPE) and its accelerated degradation with ROS. The final degraded product is soluble in body fluid.

FIG. 6B is a set of images of the hydrogel solution that is flowable and injectable at both 4° C. and 12° C., and it quickly forms a solid gel at 30° C. and 37° C.

FIG. 6C is an image showing retention of PTβR2I encapsulated in the developed hydrogel in the wound area after 24 hours, observed using IVIS, with comparative collagen gel retention results.

FIG. 6D is a graph showing the quantification of drug retention by relative ROI intensity derived from IVIS images (n=3).

FIG. 6E is a graph of the cytotoxicity of different concentrations of the degraded product to dermal fibroblasts, evaluated by MTT assay (n=4).

FIG. 6F is a pair of images showing the in vivo biocompatibility of the ROS-sensitive hydrogels, examined by F4/80 staining (green) on tissue samples in collagen gel (left), and with subcutaneously injected hydrogel after 7 days (right). Nuclei were stained with DAPI (blue).

FIG. 6G is a graph of the quantification of F4/80+ cell ratio based on the images (n=5).

FIG. 6H is a graph of in vitro release profiles of 3 different concentrations of PTβR2I in ROS-responsive gel for 21 days (n=4).

FIG. 6I is a graph of the bioactivity of PTβR2I released from ROS-responsive gel at days 3, 8, and 16 (n=5). All data are shown as mean±standard deviation. Data were analyzed by one-way ANOVA with the Bonferroni post-test.

FIG. 7A is a schematic of a timeline of diabetic mouse wound healing.

FIG. 7B is a set of representative images of wounds taken from day 0 to day 14. Wounds were created using 5 mm biopsy punches on the dorsal skin of db/db mice and treated with hydrogel and PTβR2I/Gel topically and subcutaneously.

FIG. 7C is a graph of wound sizes over a 14-day course of treatment. Wound size ratios were normalized to day 0 (n≥6).

FIG. 7D is a set of images of Cytokeratin 10 and 14 staining illustrating enhanced keratinocyte migration at days 3, 8, and 14 in the PTβR2I/Gel group, compared with the no-treatment group and gel-only group. Scale bar=500 μm for days 3 and 8. Scale bar=200 μm for day 14.

FIG. 7E is a set of Masson's trichrome stained wound sections on day 8 of no treatment, treatment with gel, and treatment with PTβR2I encapsulated in the gel. Scale bar=500 μm.

FIG. 7F is a graph of the quantification of epidermal thickness at day 8 (n=8).

FIG. 7G is a set of images of immunohistochemical (IHC) staining of K14 (red) in the wounded region at day 14. Scale bars=50 μm.

FIG. 7H is a graph of hair follicle density in the wounded area at day 14 (n=5).

FIG. 7I is a set of representative images of proliferated cells in the wound area at day 3, made using an anti-Ki67 antibody.

FIG. 7J is a set of representative images of proliferated cells in the wound area at day 8, made using an anti-Ki67 antibody.

FIG. 7K is a set of representative images of proliferated cells in the wound area at day 14 made using anti-Ki67 antibody.

FIG. 7L is a graph of the quantification of proliferated cell (Ki67+) density (n=5). All data are shown as mean±standard deviation. Data were analyzed by one-way ANOVA with Bonferroni post-test (n.s.p>0.05, ** p<0.01, *** p<0.01).

FIG. 8A is a set of images of representative day 3 immunofluorescence stain images of vessel formation, made using CD31 and α-SMA (nuclei in blue using DAPI).

FIG. 8B is a set of images of representative day 8 immunofluorescence stain images of vessel formation, made using CD31 and α-SMA (nuclei in blue using DAPI).

FIG. 8C is a set of images of representative day 14 immunofluorescence stain images of vessel formation, made using CD31 and α-SMA (nuclei in blue using DAPI).

FIG. 8D is a graph of densities of CD31+ and α-SMA+vessels formed in the wound area (n=5).

FIG. 8E is a gel angiogenesis array analysis for tissue samples collected on day 3 post-treatment.

FIG. 8F is a graph of band intensities of proteins in the angiogenesis array.

FIG. 9A is a set of representative IHC images, using anti-CD86, of tissue sections at days 3, 8, and 14 post-treatment. Nuclei were stained with DAPI. Scale bar=50 μm.

FIG. 9B is a graph of the quantification of CD86+ cell density in the wounded area (n=6). Treatment of PTβR2I via hydrogel alleviated inflammatory response by decreasing M1 macrophage density.

FIG. 9C is a set of images of the ROS content of tissue sections, revealed by CM-H2DCFDA, at 3, 8, and 14 days post-treatment. Scale bar=50 μm.

FIG. 9D is a graph of the quantification of relative ROS+ cell density (n=6).

FIG. 9E is a gel protein array analysis of pro-inflammatory cytokines for tissue samples collected on day 3 post-treatment.

FIG. 9F is a graph of a quantification summary for the cytokine array in FIG. 9E. All data are shown as mean±standard deviation.

FIG. 9G is a Western blot analysis of p-p38 in the wounded skin of db/db mice on day 3. GAPDH was used as a loading control. By day 3, PTβR2I has downregulated the expression of p-p38 and p-Smad2/3

FIG. 9H is a Western blot analysis of p-p38 in the wounded skin of db/db mice on day 8. GAPDH was used as a loading control. By day 8, it has continued to downregulate p-p38, without compromising the expressions of p-Smad2/3 and α-SMA.

FIG. 10A is a graph of α-SMA+ and CD31-myofibroblast densities (n=4) quantified from images in FIG. 9A, FIG. 9B, and FIG. 9C.

FIG. 10B is a set of images from a PSR staining study of wound sections at 8 days and 14 days post-treatment. Scale bar=50 μm.

FIG. 10C is a graph of relative total collagen contents in the wound area on days 8 and 14, based on acquired PSR images (n≥5).

FIG. 10D is a graph of ratios of collagen I to collagen III at two time points, from PSR images (n=5). The ratio for healthy skin is 26.3, indicated with a dashed line. All data demonstrated as mean±standard deviation.

FIG. 11A is a schematic of a timeline of db/+ mice wound healing.

FIG. 11B is a set of representative images of wounds of db/+ mice, taken from day 0 to day 8. Wounds were created using 5 mm biopsy punches on the dorsal skin of db/+ mice and treated with gel and PT β R2I/Gel via subcutaneous injection.

FIG. 11C is a graph of wound sizes over 8 days for each treatment. Wound size ratios were normalized to day 0 (n≥4).

FIG. 11D is an immunoblotting analysis of p-p38 and p-Smad2/3 proteins in dermal fibroblasts cultured under low glucose conditions. GAPDH was used as a loading control.

FIG. 11E is a pair of representative images of IF staining using α-SMA antibody of dermal fibroblasts cultured with 1 g/L glucose after 24 hours.

FIG. 11F is a graph of the quantification of the α-SMA+ cell ratio from the IF images (n=6) in FIG. 11E. All data demonstrated as mean±standard deviation. Data were analyzed by one-way ANOVA with Bonferroni post-test (n.s.p>0.05, *** p<0.05, ** p<0.01, *** p<0.001).

FIG. 12 is a schematic representing the mechanisms behind the wound healing of the present disclosure.

FIG. 13A is a graph from a cytotoxicity test of PTβR2I on dermal fibroblasts (n=5). Data were analyzed by one-way ANOVA with Bonferroni post-test (n.s.p>0.05).

FIG. 13B is a graph from a cytotoxicity test of PTβR2I on major skin cells, including HaCaT cells (n=4). Data were analyzed by one-way ANOVA with Bonferroni post-test (n.s.p>0.05).

FIG. 13C is a graph from a cytotoxicity test of PTβR2I on endothelial cells (n=4). Data were analyzed by one-way ANOVA with Bonferroni post-test (n.s.p>0.05).

FIG. 14A is an immunoblotting analysis of p-p38 in dermal fibroblasts cultured under high glucose levels. (n=3, *** p<0.001).

FIG. 14B is a graph from the immunoblotting analysis in FIG. 14A.

FIG. 15A is an immunoblotting of the cells treated with or without PTβR2I under high TGFβ1 conditions.

FIG. 15B is a pair of representative IF images of dermal fibroblasts cultured under high glucose conditions. All dermal fibroblasts were cultured on 2D collagen gels in 48-well plates.

### DETAILED DESCRIPTION OF THE DISCLOSURE

The present disclosure is based, at least in part, on the discovery that the inhibition of TGFβ signaling within wound sites of diabetic patients resulted in enhanced wound healing by concurrently reducing inflammation and promoting the migration of keratinocytes, fibroblasts, and endothelial cells in diabetic wounds.

In various aspects, multifunctional drug delivery systems and methods are disclosed that concurrently reduce inflammation and promote the migration of keratinocytes, fibroblasts, and endothelial cells in diabetic wounds. The enhanced endothelial cell migration resulting from the use of the disclosed drug delivery systems and methods leads to accelerated angiogenesis. The disclosed drug delivery system gradually releases a peptide-based TGFβ receptor II (TGFβRII) inhibitor, ECGLLPVGRPDRVWRLCK, also referred to herein as “ECG”, to block the TGFβ1/p38 pathway under TGFβ1 and high glucose conditions existing in the diabetic wounds. The TGFβ1/p38 pathway is directly associated with diabetic inflammation and abnormal cell migration. The non-toxic peptide-based TGFβRII inhibitor has the advantage over small organic inhibitors, as most small organic inhibitors are not suitable for wound healing due to the effective dosages of the small organic inhibitors typically falling above toxic levels.

Without being limited to any particular theory, ECG blocks TGFβ1 binding to TGFβRIIs, the initial step of the TGFβ1/p38 pathway. ECG specifically binds to the TGFβRIIs and has a higher affinity for TGFβRIIs than TGFβ1. Therefore, once the ECG binds to TGFβRIIs, TGFβ1 is prevented from binding to the TGFβRII receptors, effectively blocking the initiation of the TGFβ1/p38 pathway.

In various aspects, the drug delivery carrier includes a dual-function NIPAAm-based hydrogel that is both thermosensitive and responsive to reactive oxygen species (ROS). As demonstrated in the Examples below, the NIPAAm-based hydrogel in one embodiment was an injectable liquid at a temperature of 4° C., providing for injection into a patient using a suitable needle including, but not limited to a 26 G needle commonly used for animal surgery. At a temperature of about 37° C., representative of a typical body temperature, the hydrogel transitioned from a liquid to a solid gel within about 8 seconds, indicating excellent thermosensitivity. As demonstrated in the Examples below, several different hydrogel compositions with NIPAAm contents ranging from about 6 wt % to about 10 wt % degraded to about 50%-60% of initial weight after exposure to 50 mM H₂O₂ solution over a period of 2-4 weeks.

In various aspects, the hydrogel of the drug delivery carrier is synthesized using any suitable method without limitation. In some aspects, the hydrogel is synthesized by free radical polymerization using N-isopropylacrylamide (NIPAAm), 2-hydroxyethyl methacrylate (HEMA), and 4-(hydroxymethyl)-phenylboronic acid pinacol ester (HPPE).

### TGFβ Receptor II (TGFβRII) Inhibiting Agents

As described herein, TGFβ1 signaling has been implicated in various diseases, disorders, and conditions. As such, inhibition of TGFβ1 signaling can be used for the treatment of such conditions. A TGFβ1 signal inhibiting agent can inhibit the initial step of activating the TGFβ1/p38 pathway. TGFβ1 signal inhibition can comprise modulating binding of TGFβ1 to TGFβ receptor II (TGFβRII).

In various aspects, the TGFβ1 signal inhibiting agent can be any composition or method that can modulate the binding of TGFβ1 to TGFβRII. For example, a TGFβ1 signal inhibiting agent can be an inhibitor or an antagonist.

In one aspect, a TGFβ1 signal inhibiting agent can be a TGFβRII antibody (e.g., a monoclonal antibody to TGFβRII).

In other aspects, a TGFβ1 signal inhibiting agent can be a small molecule inhibitor of TGFβ1 signaling. As another example, a TGFβ1 signal inhibiting agent can be a short hairpin RNA (shRNA). As another example, a TGFβ1 signal inhibiting agent can be a short interfering RNA (siRNA).

As another example, RNA (e.g., long noncoding RNA (IncRNA)) can be targeted with antisense oligonucleotides (ASOs) as a therapeutic. Processes for making ASOs targeted to RNAs are well known; see e.g. Zhou et al. 2016 Methods Mol Biol. 1402:199-213. Except as otherwise noted herein, therefore, the process of the present disclosure can be carried out in accordance with such processes.

Various aspects of the present disclosure provide for targeting of TGFβ1, its receptor TGFβRII, or its downstream signaling. The present disclosure provides methods of promoting wound healing in diabetic patients, based on the discovery that a peptide-based TGFβ receptor II (TGFβRII) inhibitor can bind to TGFβRII receptors with higher affinity than TGFβ1. Further, the administration of the peptide-based TGFβ receptor II (TGFβRII) inhibitor potently reduces inflammation and promotes the migration of keratinocytes, fibroblasts, and endothelial cells in diabetic wounds.

In some aspects, the TGFβRII inhibitor is ECG, a peptide-based TGFβ receptor II (TGFβRII) inhibitor comprising a polypeptide with the amino acid sequence ECGLLPVGRPDRVWRLCK (SEQ ID NO:1) and variations thereof.

Without being limited to any particular theory, ECG blocks the initial step of the TGFβ1/p38 pathway—TGFβ1 binding to TGFβRIIs. ECG specifically binds to the TGFβRIIs with a higher affinity for TGFβRIIs than TGFβ1. Therefore, once the ECG binds to TGFβRIIs, TGFβ1 cannot bind to these receptors to initiate the TGFβ1/p38 pathway.

### Molecular Engineering

The following definitions and methods are provided to better define the present disclosure and to guide those of ordinary skill in the art in the practice of the present disclosure. Unless otherwise noted, terms are to be understood according to conventional usage by those of ordinary skill in the relevant art.

The terms “heterologous DNA sequence”, “exogenous DNA segment” or “heterologous nucleic acid,” as used herein, each refers to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling or cloning. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

A “promoter” is generally understood as a nucleic acid control sequence that directs transcription of a nucleic acid. An inducible promoter is generally understood as a promoter that mediates the transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as much as several thousand base pairs from the start site of transcription.

A “transcribable nucleic acid molecule” as used herein refers to any nucleic acid molecule capable of being transcribed into an RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754).

The “transcription start site” or “initiation site” is the position surrounding the first nucleotide that is part of the transcribed sequence, which is also defined as position+1. With respect to this site, all other sequences of the gene and its controlling regions can be numbered. Downstream sequences (i.e., further protein-encoding sequences in the 3′ direction) can be denominated positive, while upstream sequences (mostly of the controlling regions in the 5′ direction) are denominated negative.

“Operably-linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A construct of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

The term “transformation” refers to the transfer of a nucleic acid fragment into the genome of a host cell, resulting in genetically stable inheritance. Host cells containing the transformed nucleic acid fragments are referred to as “transgenic” cells and organisms comprising transgenic cells are referred to as “transgenic organisms”.

“Transformed,” “transgenic,” and “recombinant” refer to a host cell or organism such as a bacterium, cyanobacterium, animal, or plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using paired primers, nested primers, single specific primers, degenerate primers, gene-specific primers, vector-specific primers, partially mismatched primers, and the like. The term “untransformed” refers to normal cells that have not been through the transformation process.

“Wild-type” refers to a virus or organism found in nature without any known mutation.

Design, generation, and testing of the variant nucleotides, and their encoded polypeptides, having the above required percent identities and retaining a required activity of the expressed protein are within the skill of the art. For example, directed evolution and rapid isolation of mutants can be according to methods described in references including, but not limited to, Link et al. (2007) Nature Reviews 5(9), 680-688; Sanger et al. (1991) Gene 97(1), 119-123; Ghadessy et al. (2001) Proc Natl Acad Sci USA 98(8) 4552-4557. Thus, one skilled in the art could generate a large number of nucleotide and/or polypeptide variants having, for example, at least 95-99% identity to the reference sequence described herein and screen such for desired phenotypes according to methods routine in the art.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2, or Megalign (DNASTAR) software is used to align sequences. Those skilled in the art can determine appropriate parameters for measuring alignment, including any algorithms needed to achieve maximal alignment over the full length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A.

Generally, conservative substitutions can be made at any position so long as the required activity is retained. So-called conservative exchanges can be carried out in which the amino acid that is replaced has a similar property as the original amino acid, for example, the exchange of Glu by Asp, Gln by Asn, Val by 11e, Leu by 11e, and Ser by Thr. For example, amino acids with similar properties can be Aliphatic amino acids (e.g., Glycine, Alanine, Valine, Leucine, Isoleucine); Hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. The amino acid sequence can be modulated with the help of art-known computer simulation programs that can produce a polypeptide with, for example, improved activity or altered regulation. On the basis of these artificially generated polypeptide sequences, a corresponding nucleic acid molecule coding for such a modulated polypeptide can be synthesized in-vitro using the specific codon usage of the desired host cell.

“Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tm) of a DNA duplex between the two sequences. If a particular duplex has a melting temperature lower than 65° C. in the salt conditions of a 6×SSC, then the two sequences will not hybridize. On the other hand, if the melting temperature is above 65° C. in the same salt conditions, then the sequences will hybridize. In general, the melting temperature for any hybridized DNA:DNA sequence can be determined using the following formula: Tm=81.5° C.+16.6(log 10[Na+])+0.41(fraction G/C content)−0.63(% formamide)−(600/I). Furthermore, the Tm of a DNA:DNA hybrid is decreased by 1-1.5° C. for every 1% decrease in nucleotide identity (see e.g., Sambrook and Russel, 2006).

Host cells can be transformed using a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transfected cells can be selected and propagated to provide recombinant host cells that comprise the expression vector stably integrated in the host cell genome.

Conservative Substitutions I

| Side Chain Characteristic | Amino Acid |
|---|---|
| Aliphatic Non-polar | G A P I L V |
| Polar-uncharged | C S T M N Q |
| Polar-charged | D E K R |
| Aromatic | H F W Y |
| Other | N Q D E |

Conservative Substitutions II

| Side Chain Characteristic | Amino Acid |
|---|---|
| Non-polar (hydrophobic) | |
| A. Aliphatic: | A L I V P |
| B. Aromatic: | F W |
| C. Sulfur-containing: | M |
| D. Borderline: | G |
| Uncharged-polar | |
| A. Hydroxyl: | S T Y |
| B. Amides: | N Q |
| C. Sulfhydryl: | C |
| D. Borderline: | G |
| Positively Charged (Basic): | K R H |
| Negatively Charged (Acidic): | D E |

Conservative Substitutions III

| Original Residue | Exemplary Substitution |
|---|---|
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, Ala, Phe, |
| Leu (L) | lle, Val, Met, Ala, Phe |
| Lys (K) | Arg, Gln, Asn |
| Met(M) | Leu, Phe, Ile |
| Phe (F) | Leu, Val, Ile, Ala |
| Pro (P) | Gly |
| Ser (S) | Thr |
| Thr (T) | Ser |
| Trp(W) | Tyr, Phe |
| Tyr (Y) | Trp, Phe, Tur, Ser |
| Val (V) | Ile, Leu, Met, Phe, Ala |

Exemplary nucleic acids which may be introduced to a host cell include, for example, DNA sequences or genes from another species, or even genes or sequences which originate with or are present in the same species, but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA that is already present in the cell, DNA from another individual of the same type of organism, DNA from a different organism, or DNA generated externally, such as a DNA sequence containing an antisense message of a gene, or a DNA sequence encoding a synthetic or modified version of a gene.

Host strains developed according to the approaches described herein can be evaluated by a number of means known in the art (see e.g., Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Methods of down-regulation or silencing genes are known in the art. For example, expressed protein activity can be down-regulated or eliminated using antisense oligonucleotides (ASOs), protein aptamers, nucleotide aptamers, and RNA interference (RNAi) (e.g., small interfering RNAs (siRNA), short hairpin RNA (shRNA), and micro RNAs (miRNA) (see e.g., Rinaldi and Wood (2017) Nature Reviews Neurology 14, describing ASO therapies; Fanning and Symonds (2006) Handb Exp Pharmacol. 173, 289-303G, describing hammerhead ribozymes and small hairpin RNA; Helene, et al. (1992) Ann. N.Y. Acad. Sci. 660, 27-36; Maher (1992) Bioassays 14(12): 807-15, describing targeting deoxyribonucleotide sequences; Lee et al. (2006) Curr Opin Chem Biol. 10, 1-8, describing aptamers; Reynolds et al. (2004) Nature Biotechnology 22(3), 326-330, describing RNAi; Pushparaj and Melendez (2006) Clinical and Experimental Pharmacology and Physiology 33(5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, TX; Sigma Aldrich, MO; Invitrogen). Several siRNA molecule design programs using a variety of algorithms are known to the art (see e.g., Cenix algorithm, Ambion; BLOCK-iT™ RNAi Designer, Invitrogen; siRNA Whitehead Institute Design Tools, Bioinformatics & Research Computing). Traits influential in defining optimal siRNA sequences include G/C content at the termini of the siRNAs, Tm of specific internal domains of the siRNA, siRNA length, position of the target sequence within the CDS (coding region), and nucleotide content of the 3′ overhangs.

### Genome Editing

As described herein, TGFβ1 signaling can be modulated (e.g., reduced, eliminated, or enhanced) using genome editing. Processes for genome editing are well known; see e.g. Aldi 2018 Nature Communications 9(1911). Except as otherwise noted herein, therefore, the process of the present disclosure can be carried out in accordance with such processes.

For example, genome editing can comprise CRISPR/Cas9, CRISPR-Cpf1, TALEN, or ZNFs. Adequate blockage of TGFβ1 signaling by genome editing can result in enhanced wound healing in diabetic patients,

As an example, clustered regularly interspaced short palindromic repeats (CRISPR)/CRISPR-associated (Cas) systems are a new class of genome-editing tools that target desired genomic sites in mammalian cells. Recently published type II CRISPR/Cas systems use Cas9 nuclease that is targeted to a genomic site by complexing with a synthetic guide RNA that hybridizes to a 20-nucleotide DNA sequence and immediately preceding an NGG motif recognized by Cas9 (thus, a (N)20NGG target DNA sequence). This results in a double-strand break three nucleotides upstream of the NGG motif. The double strand break instigates either non-homologous end-joining, which is error-prone and conducive to frameshift mutations that knock out gene alleles, or homology-directed repair, which can be exploited with the use of an exogenously introduced double-strand or single-strand DNA repair template to knock in or correct a mutation in the genome. Thus, genomic editing, for example, using CRISPR/Cas systems could be useful tools for therapeutic applications for the TGFβ1 signaling inhibitor to target cells by the removal of TGFβ1 signals.

For example, the methods as described herein can comprise a method for altering a target polynucleotide sequence in a cell comprising contacting the polynucleotide sequence with a clustered regularly interspaced short palindromic repeats-associated (Cas) protein.

### Formulation

The agents and compositions described herein can be formulated by any conventional manner using one or more pharmaceutically acceptable carriers or excipients as described in, for example, Remington's Pharmaceutical Sciences (A.R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Maryland, 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutically active substances is well known in the art (see generally Remington's Pharmaceutical Sciences (A.R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60 ºC, for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic, or other physical forces.

Controlled-release (or sustained-release) preparations may be formulated to extend the activity of the agent(s) and reduce dosage frequency. Controlled-release preparations can also be used to affect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for the treatment of the disease, disorder, or condition.

### Therapeutic Methods

Also provided is a process promoting wound healing in a diabetic patient in need by administration of a therapeutically effective amount of TGFβ receptor II (TGFβRII) inhibitor so as to decrease inflammation and promote migration of keratinocytes, fibroblasts, and endothelial cells to the wound site, thereby promoting angiogenesis and epidermis regeneration associated with wound closure.

Methods described herein are generally performed on a subject in need thereof. A subject in need of the therapeutic methods described herein can be a subject having, diagnosed with, suspected of having, or at risk for developing a diabetes-related wound. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans or chickens. For example, the subject can be a human subject.

Generally, a safe and effective amount of the TGFβ receptor II (TGFβRII) inhibitor is, for example, an amount that would cause the desired therapeutic effect in a subject while minimizing undesired side effects. In various embodiments, an effective amount of the TGFβ receptor II (TGFβRII) inhibitor described herein can substantially promote wound healing by inhibiting inflammatory cytokine production and promoting cell migrations associated with angiogenesis and epidermal growth.

According to the methods described herein, the administration is accomplished using a thermally sensitive hydrogel composition loaded with a therapeutically effective amount of the TGFβ receptor II (TGFβRII) inhibitor as described herein.

When used in the treatments described herein, a therapeutically effective amount of the TGFβ receptor II (TGFβRII) inhibitor can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to promote the healing of diabetes-related wounds.

The amount of a composition described herein that can be combined with a pharmaceutically acceptable carrier to produce a single dosage form will vary depending upon the subject or host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

The toxicity and therapeutic efficacy of compositions described herein can be determined by standard pharmaceutical procedures in cell cultures or experimental animals for determining the LD50 (the dose lethal to 50% of the population) and the ED50, (the dose therapeutically effective in 50% of the population). The dose ratio between toxic and therapeutic effects is the therapeutic index that can be expressed as the ratio LD50/ED50, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4th ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from the compositions and methods described herein. Generally, treating a state, disease, disorder, or condition includes preventing, reversing, or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or a physician.

Administration of the TGFβ receptor II (TGFβRII) inhibitor can occur as a single event or over a time course of treatment. For example, the TGFβ receptor II (TGFβRII) inhibitor can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to, concurrent with, or after conventional treatment modalities for diabetes-related wounds.

The TGFβ receptor II (TGFβRII) inhibitor can be administered simultaneously or sequentially with another agent, such as an antibiotic, an anti-inflammatory, or another agent. For example, the TGFβ receptor II (TGFβRII) inhibitor can be administered simultaneously with another agent, such as an antibiotic or an anti-inflammatory. Simultaneous administration can occur through the administration of separate compositions, each containing one or more of the TGFβ receptor II (TGFβRII) inhibitor, an antibiotic, an anti-inflammatory, or another agent. Simultaneous administration can occur through the administration of one composition containing two or more of the TGFβ receptor II (TGFβRII) inhibitor, an antibiotic, an anti-inflammatory, or another agent. The TGFβ receptor II (TGFβRII) inhibitor can be administered sequentially with an antibiotic, an anti-inflammatory, or another agent. For example, the TGFβ receptor II (TGFβRII) inhibitor can be administered before or after the administration of an antibiotic, an anti-inflammatory, or another agent.

### Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal.

Agents and compositions described herein can be administered in a variety of methods well-known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency, improve the taste of the product; or improve the shelf life of the product.

In various aspects, the TGFβ receptor II (TGFβRII) inhibitor is encapsulated and administered in a carrier delivery system that is a thermally responsive hydrogel. In some aspects, the thermally-responsive hydrogel is a dual-function NIPAAm-based hydrogel that is both thermosensitive and responsive to reactive oxygen species (ROS). In some aspects, the hydrogel is synthesized by free radical polymerization using N-isopropylacrylamide (NIPAAm), 2-hydroxyethyl methacrylate (HEMA), and 4-(hydroxymethyl)-phenylboronic acid pinacol ester (HPPE).

### Screening

Also provided are methods for screening.

The subject methods find use in the screening of a variety of different candidate molecules (e.g., potentially therapeutic candidate molecules). Candidate substances for screening according to the methods described herein include, but are not limited to, fractions of tissues or cells, nucleic acids, polypeptides, siRNAs, antisense molecules, aptamers, ribozymes, triple helix compounds, antibodies, and small (e.g., less than about 2000 mw, or less than about 1000 mw, or less than about 800 mw) organic molecules or inorganic molecules including but not limited to salts or metals.

Candidate molecules encompass numerous chemical classes, for example, organic molecules, such as small organic compounds having a molecular weight of more than 50 and less than about 2,500 Daltons. Candidate molecules can comprise functional groups necessary for structural interaction with proteins, particularly hydrogen bonding, and typically include at least an amine, carbonyl, hydroxyl, or carboxyl group, and usually at least two of the functional chemical groups. The candidate molecules can comprise cyclical carbon or heterocyclic structures and/or aromatic or polyaromatic structures substituted with one or more of the above functional groups.

A candidate molecule can be a compound in a library database of compounds. One of skill in the art will be generally familiar with, for example, numerous databases for commercially available compounds for screening (see e.g., ZINC database, UCSF, with 2.7 million compounds over 12 distinct subsets of molecules; Irwin and Shoichet (2005) J Chem Inf Model 45, 177-182). One of skill in the art will also be familiar with a variety of search engines to identify commercial sources or desirable compounds and classes of compounds for further testing (see e.g., ZINC database; eMolecules.com; and electronic libraries of commercial compounds provided by vendors, for example: ChemBridge, Princeton BioMolecular, Ambinter SARL, Enamine, ASDI, Life Chemicals etc.).

Candidate molecules for screening according to the methods described herein include both lead-like compounds and drug-like compounds. A lead-like compound is generally understood to have a relatively smaller scaffold-like structure (e.g., molecular weight of about 150 to about 350 kD) with relatively fewer features (e.g., less than about 3 hydrogen donors and/or less than about 6 hydrogen acceptors; hydrophobicity character xlogP of about −2 to about 4) (see e.g., Angewante (1999) Chemie Int. ed. Engl. 24, 3943-3948). In contrast, a drug-like compound is generally understood to have a relatively larger scaffold (e.g., molecular weight of about 150 to about 500 kD) with relatively more numerous features (e.g., less than about 10 hydrogen acceptors and/or less than about 8 rotatable bonds; hydrophobicity character xlogP of less than about 5) (see e.g., Lipinski (2000) J. Pharm. Tox. Methods 44, 235-249). Initial screening can be performed with lead-like compounds.

When designing a lead from spatial orientation data, it can be useful to understand that certain molecular structures are characterized as being “drug-like”. Such characterization can be based on a set of empirically recognized qualities derived by comparing similarities across the breadth of known drugs within the pharmacopoeia. While it is not required for drugs to meet all, or even any, of these characterizations, it is far more likely for a drug candidate to meet with clinical success if it is drug-like.

Several of these “drug-like” characteristics have been summarized into the four rules of Lipinski (generally known as the “rules of fives” because of the prevalence of the number 5 among them). While these rules generally relate to oral absorption and are used to predict the bioavailability of compounds during lead optimization, they can serve as effective guidelines for constructing a lead molecule during rational drug design efforts such as may be accomplished by using the methods of the present disclosure.

The four “rules of five” state that a candidate drug-like compound should have at least three of the following characteristics: (i) weight less than 500 Daltons; (ii) a log of P less than 5; (iii) no more than 5 hydrogen bond donors (expressed as the sum of OH and NH groups); and (iv) no more than 10 hydrogen bond acceptors (the sum of N and O atoms). Also, drug-like molecules typically have a span (breadth) of between about 8 Å to about 15 Å.

### Kits

Also provided are kits. Such kits can include an agent or composition described herein and, in certain embodiments, instructions for administration. Such kits can facilitate the performance of the methods described herein. When supplied as a kit, the different components of the composition can be packaged in separate containers and admixed immediately before use. Components include, but are not limited to the peptide-based TGFβ receptor II (TGFβRII) inhibitor and the NIPAAm-based hydrogel as described herein. In some aspects, the components may include the NIPAAm-based hydrogel loaded with the therapeutically effective amount of the peptide-based TGFβ receptor II (TGFβRII) inhibitor. In other aspects, the components may include at least one or more separate ingredients used to synthesize the loaded hydrogel including, but not limited to, the peptide-based TGFβ receptor II (TGFβRII) inhibitor such as ECG, N-isopropylacrylamide (NIPAAm), 2-hydroxyethyl methacrylate (HEMA), and 4-(hydroxymethyl)-phenylboronic acid pinacol ester (HPPE). Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing the activity of the components.

Kits may also include reagents in separate containers such as, for example, sterile water or saline to be added to a lyophilized active component packaged separately. For example, sealed glass ampules may contain a lyophilized component and in a separate ampule, sterile water and/or sterile saline each of which has been packaged under a neutral non-reacting gas, such as nitrogen. Ampules may consist of any suitable material, such as glass, organic polymers, such as polycarbonate, polystyrene, ceramic, metal, or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium or video. Detailed instructions may not be physically associated with the kit; instead, a user may be directed to an Internet website specified by the manufacturer or distributor of the kit.

A control sample or a reference sample as described herein can be a sample from a healthy subject. A reference value can be used in place of a control or reference sample, which was previously obtained from a healthy subject or a group of healthy subjects. A control sample or a reference sample can also be a sample with a known amount of a detectable compound or a spiked sample.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing from the scope of the present disclosure defined in the appended claims. Furthermore, it should be appreciated that all examples in the present disclosure are provided as non-limiting examples.

### Examples

The following non-limiting examples are provided to further illustrate the present disclosure. It should be appreciated by those of skill in the art that the techniques disclosed in the examples that follow represent approaches the inventors have found function well in the practice of the present disclosure and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1: Characterization of NIPAAM-Based Hydrogels

To characterize the properties of the NIPAAm-based hydrogels disclosed herein, the following experiments were conducted.

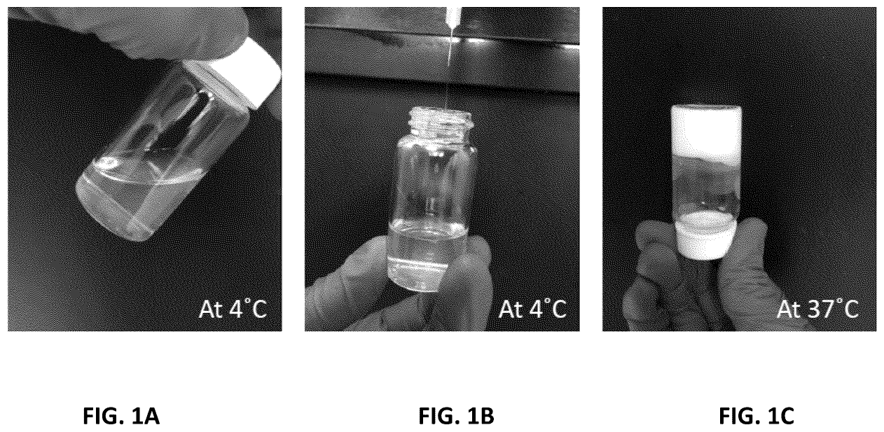

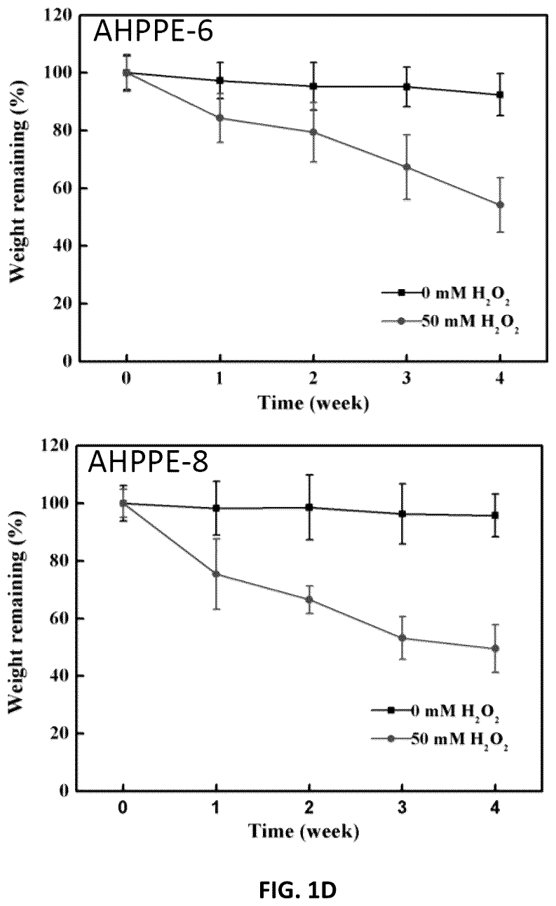

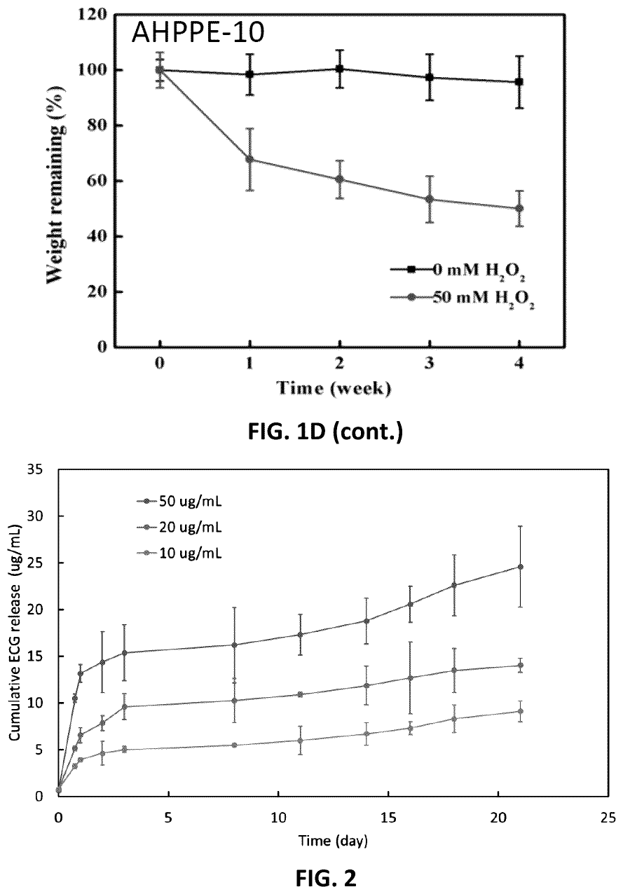

Samples of the NIPAAm-based hydrogels were synthesized by free radical polymerization as described herein. FIG. 1A shows the hydrogel in a liquid state at a temperature of 4° C. As shown in FIG. 1B, the liquid hydrogel in the liquid state at 4° C. was injectable through a 26 G needle commonly used for animal surgery. After heating the sample to about 37° C., a temperature representative of body temperature, the hydrogel transitioned from a liquid to a solid gel within about 8 seconds, indicating excellent thermosensitivity, as shown in FIG. 1C.

To characterize the degradation of the hydrogel in the presence of reactive oxygen species (ROS), three different hydrogel compositions with varying wt % of gel were synthesized: AHPPE-6 (6 wt %), AHPPE-8 (8 wt %), and AHPPE-10 (10 wt %). All samples were stored in a control solution (0 mM H₂O₂) or a solution containing 50 mM H₂O₂ for a period of four weeks. The samples were removed from the solution and weighed weekly to track the degradation of the hydrogel. As summarized in FIG. 1D, all hydrogel compositions remained relatively intact in the control solution and degraded to about 50%-60% of initial weight after weeks. The different hydrogel compositions resulted in slightly different decomposition rates over the first few weeks, but all compositions decomposed a comparable amount at the end of four weeks.

The results of these experiments demonstrated that the synthesized hydrogel showed excellent thermosensitivity as a solid gel formed at 37° C. within 8 seconds. In its liquid state at 4° C., the hydrogel was injectable through a 26 G needle. Additionally, the carrier showed quick responsiveness to overproduction of ROS, as the degradation rates increased dramatically with the presence of H₂O₂.

### Example 2: ECG Release Characteristics of NIPAAM-Based Hydrogels

To characterize the ECG release properties of the NIPAAm-based hydrogels disclosed herein, the following experiments were conducted. Hydrogel samples were synthesized with varying levels of ECG loading: 10 μg/mL, 20 μg/mL, and 50 μg/mL. The cumulative release of ECG from the hydrogel samples was assessed over a three-week period.

FIG. 2 summarizes the cumulative release of ECG from all samples over the three-week period. All release profiles were characterized by an initial rapid release rate over the first 3-4 days, followed by a more gradual release rate over the remainder of the three weeks.

The results of these experiments demonstrated that release characteristics of ECG from the carrier hydrogels loaded with different ECG loadings followed similar profiles characterized by an initial rapid release followed by a prolonged gradual release rate. The hydrogels with higher ECG loadings had a steeper initial release rate but comparable prolonged gradual release rates relative to hydrogels with lower ECG loadings.

### Example 3: Wound Closure Using NIPAAM-Based Hydrogels

To characterize the effects of treatment on wound closure using the NIPAAm-based hydrogels disclosed herein, the following experiments were conducted.

db/db (Diabetic, type I) mice were used as an animal wound model. Dermal wounds were created using sterile 5 mm biopsy punches to expose the underlying muscle fascia, followed by subcutaneous injection of either control gel (6 wt %) or ECG/gel (50 μg/mL ECG in 6 wt % gel). In addition, a subgroup of the dermally-wounded mice was left untreated as controls. The dermal wounds of all mice were observed every other day until recovery using a digital camera to record successive images as well as measuring wound size using digital calipers. Image analysis using ImageJ was also performed to do a blind measurement of wound size. Subgroups of the mice were sacrificed at three different time points: day 3, day 8, and day 16. Skin samples were collected from the sacrificed mice for histology, immunohistochemistry analysis, and Western blot studies.

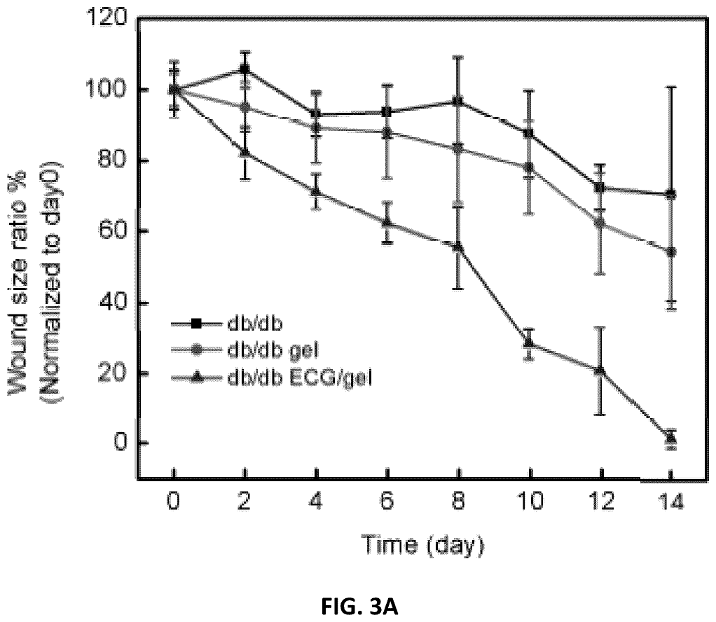

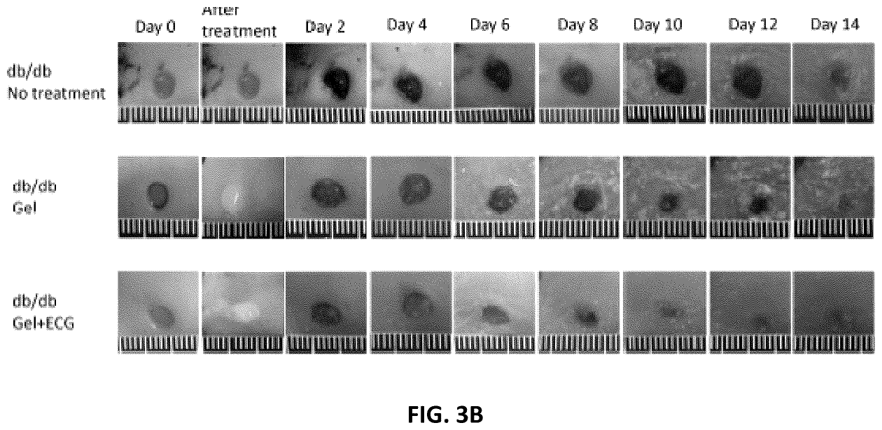

FIG. 3B contains a series of representative images of the dermal wounds of the untreated mice (No treatment, top row), mice treated with control gel (Gel, middle row), and mice treated with ECG/gel (Gel+ECG, bottom row). FIG. 3A is a graph summarizing the wound size ratios (normalized to day 0) of the mice measured from the camera images using ImageJ.

As shown in FIG. 3A, the wounds of mice treated with the control gel healed slightly more over the 14-day observation period, but the mice treated with the ECG/gel healed almost completely over the same observation period.

The immunohistochemistry and Western blot studies performed on the skin samples showed that blood vessel density and mature blood vessel density increased significantly, up to 4-fold in the group with ECG treatment compared to control at day 8, providing evidence of ECG-induced angiogenesis. In addition, neither the control gel nor ECG/gel groups exhibited any substantial inflammation. On the contrary, the density of M1 phenotype macrophages was downregulated and the density of M2 phenotype macrophages was upregulated. M1 and M2 phenotype macrophages have distinct functions; M1 activation is associated with initiating and sustaining immune responses and M2 activation is related to cell survival and proliferation. Further, the administration of ECG/gel promoted keratinocyte migration from surrounding tissues to the wound site, accelerating the rate of epidermis regeneration.

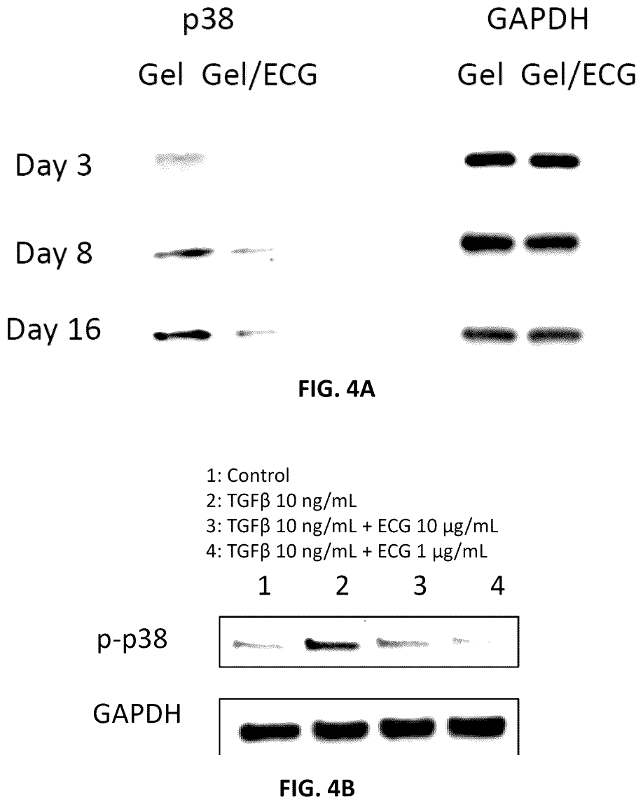

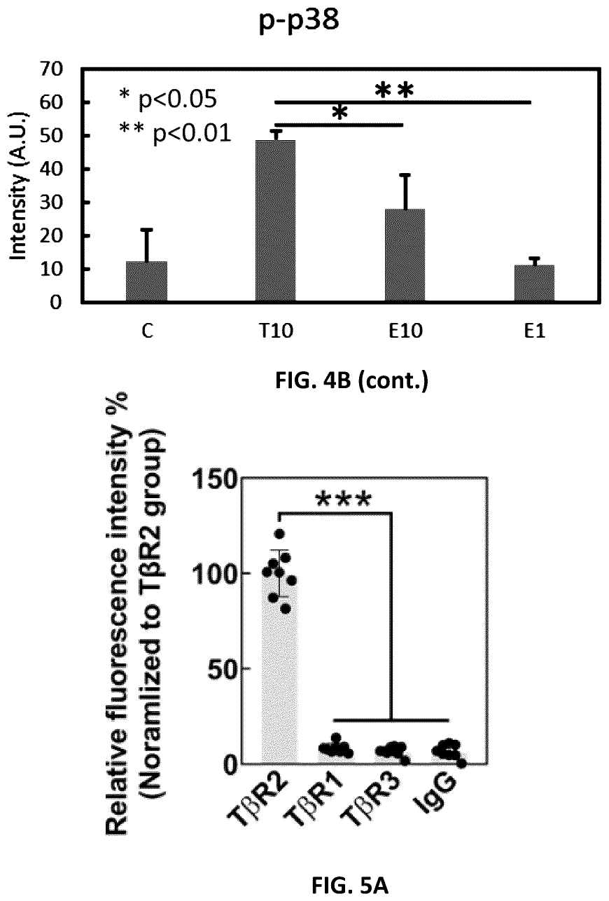

FIG. 4A is a Western blot of p38 and GAPDH (control) expression in vivo for the control gel treatment (Gel) and the ECG/Gel treatment (Gel/ECG). ECG treatment suppressed p38 expression in the wound tissues of the mice.

The results of the animal studies of these experiments demonstrated that treatment with ECG/gel significantly promoted wound closure compared to the no-treatment group or gel-only group. Scarless wound healings were observed within 16 days post-surgery within the ECG/gel treatment group. In addition, Western blot and immunohistochemistry analysis of wound tissue samples indicated that ECG treatment enhanced angiogenesis, reduced inflammation, and potentiated keratinocyte migration from surrounding tissues to the wound site to accelerate epidermis regeneration.

### Example 4: Effects of NIPAAM-Based Hydrogels on Cell Migration

To characterize the effects of ECG treatment on cell survival and migration, as well as protein expression, the following experiments were conducted.

Human dermal fibroblasts (HDF), human skin keratinocytes (HaCaT), human artery endothelial cells (HAECs), and mouse bone marrow-derived macrophages (BMM) were used to test the in vitro survival, migration, secretion of inflammatory cytokines, and angiogenic growth factors in the presence of TGF-beta I and high glucose content.

As illustrated in FIG. 4A, p38 was downregulated with ECG treatment. FIG. 4B is a Western blot of p38 and GAPDH (control) expression for dermal fibroblasts under high glucose levels. Untreated cells (control) were compared with cells exposed to 10 ng/ml of TGFβ with ECG treatments of 0 μg/mL, 10 μg/mL, and 1 μg/mL. FIG. 4B shows the Western blot (top) and a graph (bottom) summarizing p38 protein expressions by the dermal fibroblasts under the various treatments.

The results of these experiments demonstrated that ECG treatment improved the survival and migration of keratinocytes, fibroblasts, and endothelial cells in the presence of TGF-beta I. In addition, ECG suppressed pro-inflammatory cytokines expression and promoted angiogenic growth factor secretions in various cell types.

### Example 5: Thermosensitive and Antioxidant Wound Dressings Capable of Differentially Regulating TGFβ Pathways Promote Diabetic Wound Healing

Impaired wound healing in patients with diabetes poses a lifetime risk that can lead to limb amputations. While various therapies have been utilized for the treatment of diabetic wounds, current regimens do not efficiently address the key intrinsic causes of slow wound healing in the setting of diabetes, i.e., chronic inflammation, abnormal skin cell functions (particularly migration), and delayed angiogenesis. To address this persistent clinical gap, a wound dressing was developed that contains both a peptide-based TGFβ receptor II inhibitor (PTβR2I) and a thermosensitive hydrogel that scavenges reactive oxygen species (ROS). Under hyperglycemia, the PTβR2I that is gradually released from the hydrogel continuously inhibits the TGFβ1/p38 pathway, improving cell migration and decreasing tissue inflammation. The enhanced endothelial cell migration leads to faster morphogenesis. The hydrogel's ability to scavenge upregulated ROS in diabetic wounds further decreases inflammation. Meanwhile, the PTβR2I does not affect the TGFβ1/Smad2/3 pathway that is required to regulate myofibroblasts, a cell type that is critical for effective wound healing. The thermosensitive wound dressing can quickly solidify on the wounds, largely retaining the encapsulated PTβR2I. Single-dose application of the wound dressing into excisional wounds in young diabetic mice significantly accelerated wound healing, with complete wound closure by day 14, which is much faster than achieved by either solely hydrogel-treated or untreated controls. We demonstrate that the wound dressings promoted re-epithelialization, granulation, and angiogenesis while reducing excessive and deleterious inflammatory response and oxidative stress. These findings suggest that the newly developed wound dressing can provide a promising and effective wound treatment strategy in patients with diabetes.

Diabetes affects more than 34 million people in the United States alone. Elderly individuals represent a large fraction of this population, with approximately 25% of people over the age of 65 reported to have diabetes. This is currently the leading cause of non-traumatic lower limb amputation, predominantly due to the development of chronic foot wounds that are prone to infection and severe tissue damage. Various therapies have been explored to treat diabetic wounds, such as hyperbaric oxygen treatment, cell therapy, growth factor delivery, and various types of wound dressing. However, current treatments and therapies are not efficient in resolving chronic inflammation, delayed angiogenesis, and skin cell dysfunctions (particularly migration), which are key intrinsic barriers to diabetic wound healing.

To address these causes, control of TGFβ signaling is crucial because it plays direct and differential roles in delaying or promoting diabetic wound healing. The TGFβ1/p38 pathway is directly associated with prolonged tissue inflammation and impaired skin cell migration. Additionally, reduced endothelial cell migration leads to delayed angiogenesis. Meanwhile, TGFβ1/Smad2/3 pathway is required to regulate myofibroblasts, which are critical cells for wound healing. If the TGFβ1/p38 pathway is inhibited without impacting the TGFβ1/Smad2/3 pathway, this would presumably accelerate diabetic wound healing. However, current approaches have not been able to achieve this goal, and new treatments are critically needed.

To reduce the deleterious impact of TGFβ1 on diabetic wound healing, systemic delivery of TGFβ inhibitors or anti-TGFβ antibodies have been commonly used to decrease the amount of active TGFβ1. While these approaches have shown some therapeutic benefits, relatively low efficacy and bioavailability have hindered their widespread clinical adoption. The low efficacy may reflect the fact that these inhibitors and antibodies only decrease the amount of active TGFβ1, but cannot fundamentally inhibit the TGFβ1/p38 pathway. On the other hand, TGFβ receptor (TβR) inhibitors have the potential to fundamentally inhibit the TGFβ1/p38 pathway by blocking TGFβ1 from binding to TβRs on cells. While various TβR inhibitors exist, most are used for cancer therapy. One potential challenge in using these inhibitors for diabetic wound healing is their relatively high toxicity, because the effective dosages may be above the toxic level. In addition, it is essential that the TβR inhibitors for diabetic wound healing inhibit the TGFβ1/p38 pathway while not interfering with the essential TGFβ1/Smad2/3 pathway. Nonetheless, collective preclinical studies and clinical trials suggest that targeting TGFβ signaling remains an important therapeutic strategy in accelerating diabetic wound healing.

To block the TGFβ1/p38 pathway in order to simultaneously promote cell migration and decrease inflammation in diabetic wounds, we developed a wound dressing based on a peptide-based TβRII inhibitor (PTβR2I), and an injectable, thermosensitive, fast-gelling, and ROS-scavenging hydrogel. Applied topically or by injection, the wound dressing quickly solidifies to retain the drug on the wound bed, where it gradually releases PTβR2I to continuously block the TGFβ1/p38 pathway and enhance skin cell migration, stimulate angiogenesis, and attenuate tissue inflammation. The hydrogel can scavenge ROS to reduce wound inflammation. Here, it is demonstrated that PTβR2I inhibits the TGFβ1/p38 pathway under high glucose conditions without substantially impacting the TGFβ1/Smad2/3 pathway, which is responsible for the formation of myofibroblasts.

### The High TBR2 Binding Affinity of PTβR2I Increased Skin Cell Proliferation and Migration Under High Glucose and TGFβ1 Conditions.

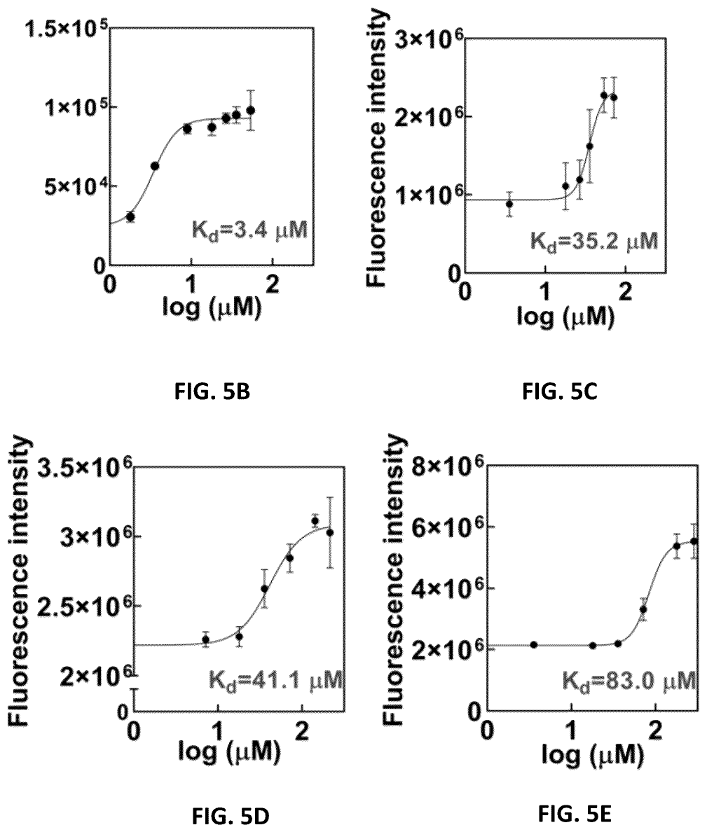

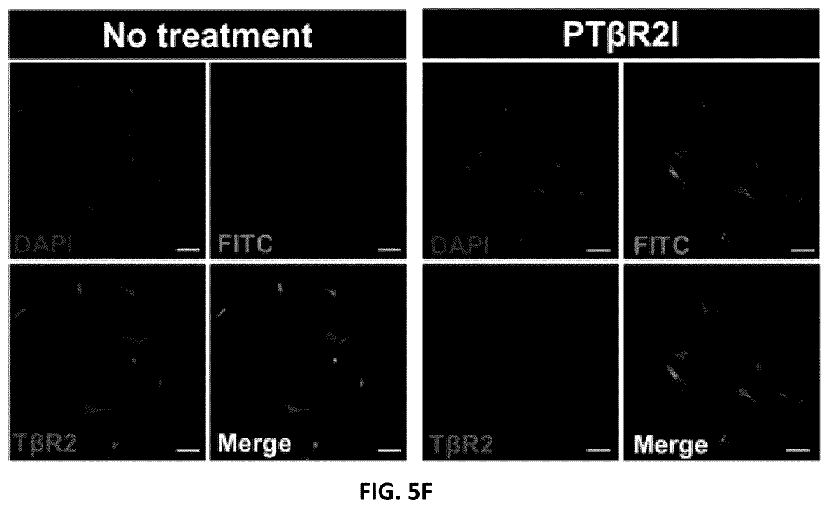

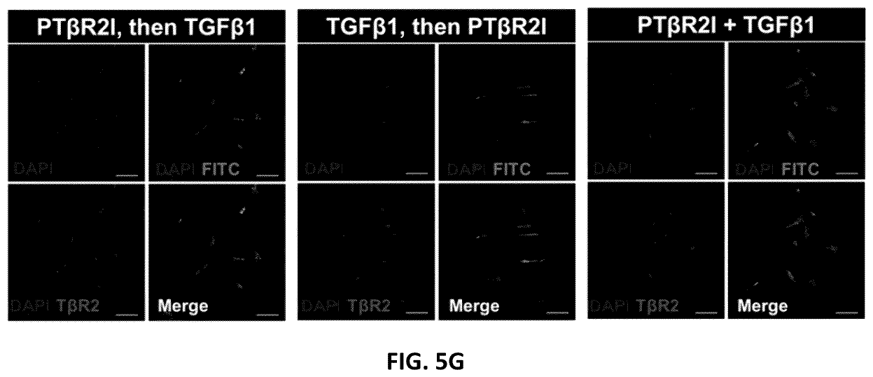

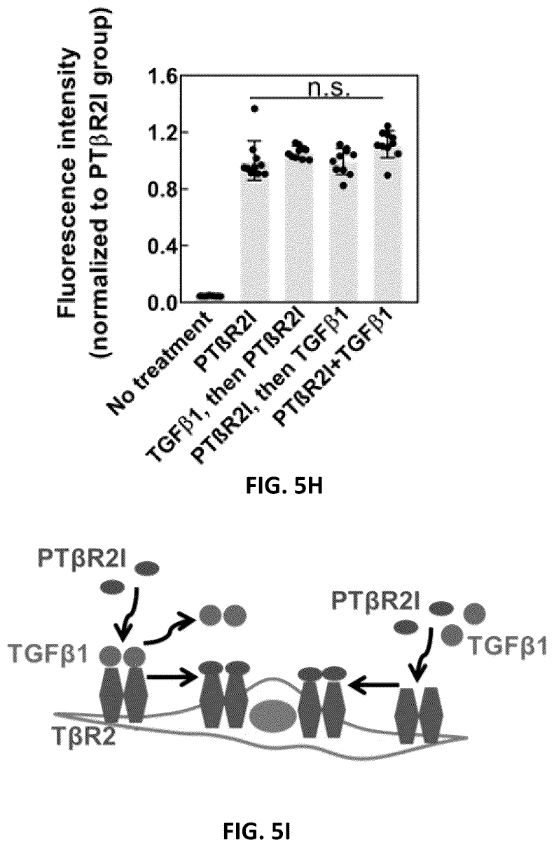

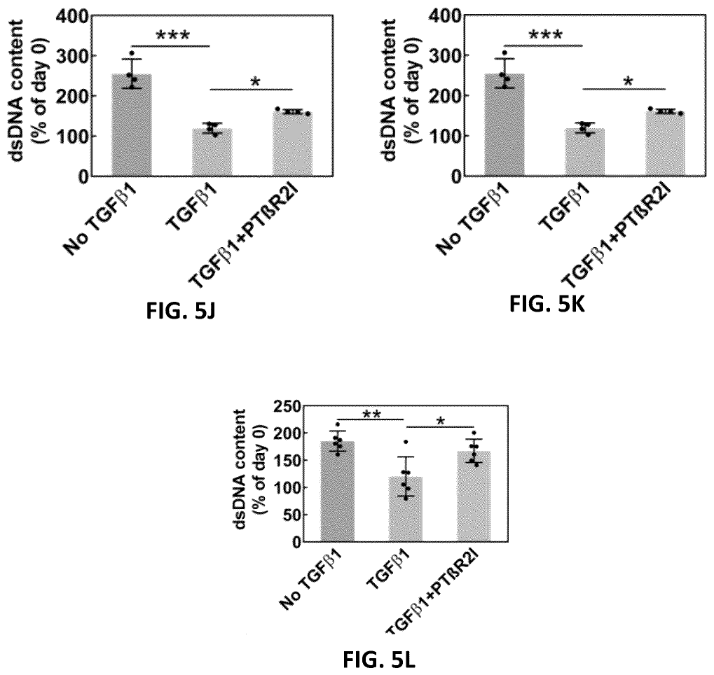

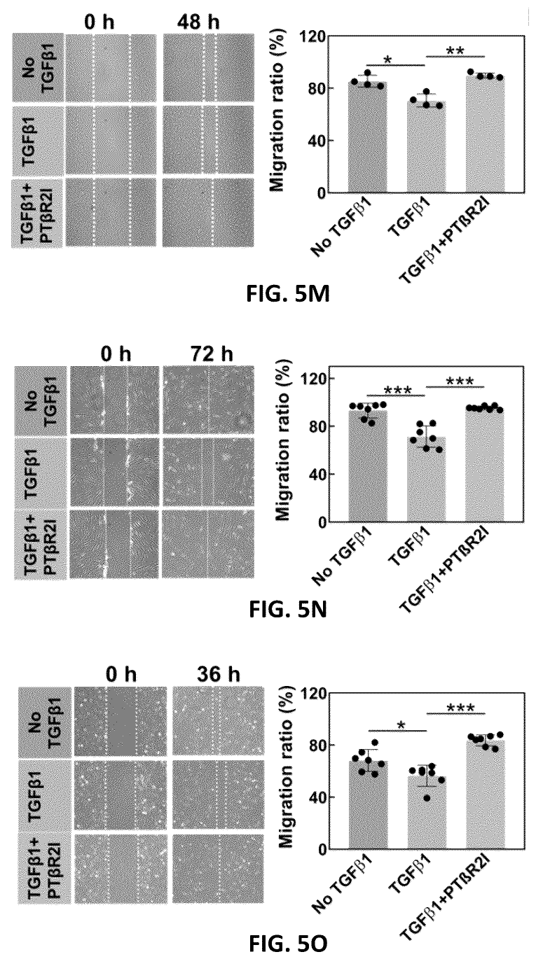

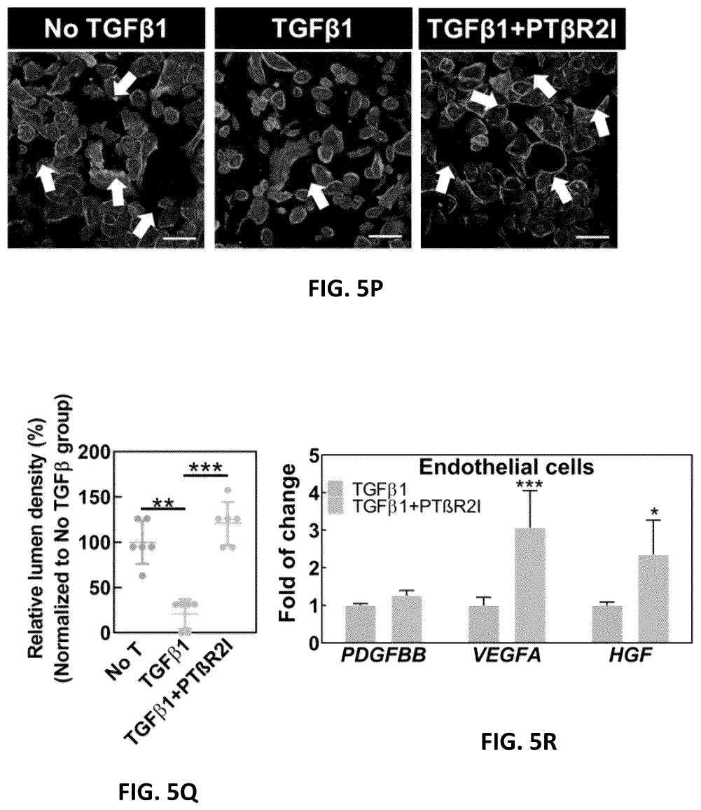

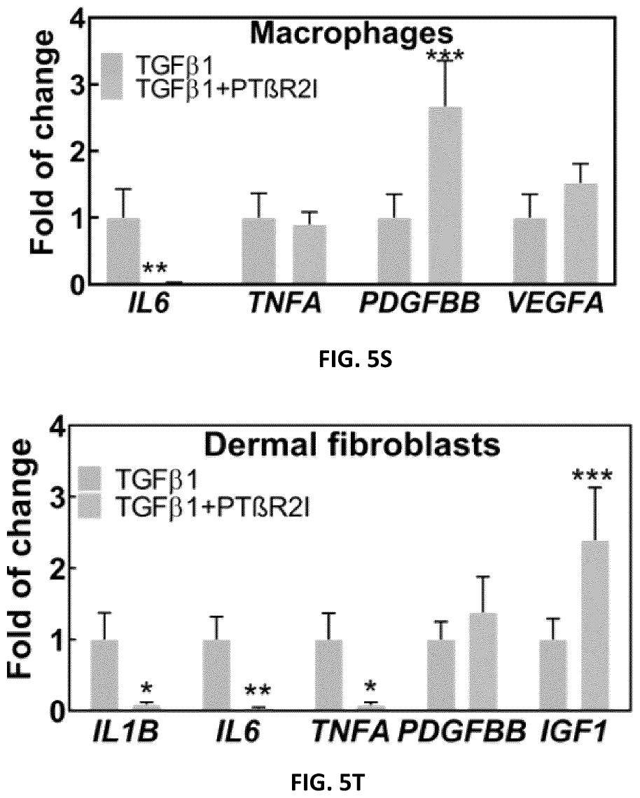

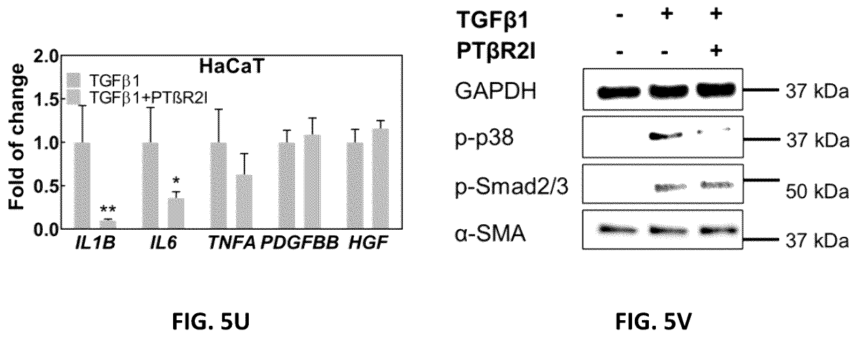

We synthesized a peptide ECGLLPVGRPDRVWRLCK-FITC (PTβR2I), based on the sequence from a phage display library that binds specifically to TβR2I and interactions between TβR2 and TGFβ1.63-65 We first examined the specific binding of PTβR2I with TβR2 using an ELISA-like binding assay. TGFβ receptor I (TβR1), TGFβ receptor III (TβR3), and IgG were used as controls. We confirmed that PTβR2I has remarkably higher binding affinity to TβR2 than its binding affinities to TβR1, TβR3, or IgG (p<0.001 FIG. 5A). The dissociation constant (Kd) of the PTβR2I binding to TβR2 is 3.4 μM, more than 10 times lower than those for PTβR2I binding to TβR1, TβR3, and IgG (FIG. 5B-E). This finding was further validated on the cellular level. We used human dermal fibroblasts (HDFs) to study the binding affinity of PTβR2I to TβR2 under a glucose level of 4.5 g/L, which mimics the hyperglycemic conditions of diabetes. With PTβR2I added, the HDF binding sites on HDF of TβR2 were occupied, so the TβR2 could not be detected by immunofluorescence (FIG. 5F).

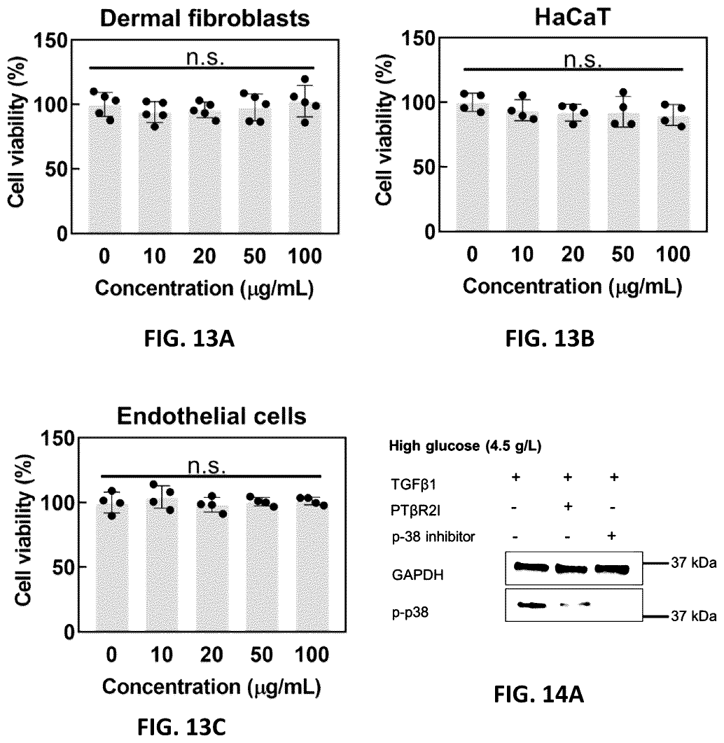

A competitive binding test between PTβR2I and TGFβ1 was also performed at the cellular level. We found that the fluorescence of TβR2 was not detected with PTβR2I treatments, either pre-, post-, or simultaneous to TGFβ1 treatments (FIG. 5G). The fluorescent intensities of FITC-labeled PTβR2I were consistent whether PTβR2I or TGFβ1 was first bound to TβR2 (FIG. 5H). These results demonstrated that PTβR2I has a higher binding affinity to TβR2 than TGFβ1. Once PTβR2I binds to TβR2, TGFβ1 cannot bind to the receptor. More interestingly, PTβR2I is able to pull off the bound TGFβ1 from the receptors and then competitively occupy the binding sites instead (FIG. 5I). In addition, the cytotoxicity of PTβR2I was tested in different concentrations on several major types of cells, including HDF, human aortic endothelial cells (HAEC), and human keratinocyte HaCaT cells (FIG. 13A-C). With PTβR2I treatment, those cells showed no decrease in viability at the test concentrations (1 to 100 g/mL), indicating high cytocompatibility and biotolerance to PTβR2I.

During diabetic wound healing, keratinocytes, fibroblasts, and endothelial cells are respectively involved in re-epithelialization, dermal formation, and angiogenesis, yet their proliferation and migration are compromised in the diseased environment due to the high glucose concentration and upregulated TGFβ. Whether PTβR2I can restore the proliferation and migration of keratinocytes, fibroblasts, and endothelial cells under these conditions was investigated. Significantly improved proliferation of HaCaTs, HDFs, and HAECs were found in the groups with PTβR2I (FIG. 1j-l, p<0.05 for HaCaTs, p<0.001 for HDFs, and p<0.05 for HEACs). To determine how PTβR2I influences the migration of HDFs, HaCaTs, and HAECs under high glucose and TGFβ1 conditions, a 2D scratch assay was used. TGFβ1 significantly decreased the migration of all three cell types (FIG. 5M-O). After the cells were treated with PTβR2I, a significantly higher number of cells had migrated. These results demonstrate that PTβR2I treatment can significantly increase the proliferation and migration of keratinocytes, fibroblasts, and endothelial cells.

### PTβR2I Increased Endothelial Morphogenesis and the Expression of Angiogenic Growth Factors in Skin Cells and Macrophages Under High Glucose and TGFβ1 Conditions.

In diabetic wounds, angiogenesis is delayed due to impaired endothelial morphogenesis and impeded expression of angiogenic growth factors such as vascular endothelial growth factor (VEGF), platelet-derived growth factor-BB (PDGFBB), and hepatocyte growth factor (HGF). The effect of PTβR2I treatment on endothelial morphogenesis was first examined by performing an in vitro lumen formation assay using HAECs. The lumen density in the TGFβ1 group was much reduced from that in the group without TGFβ1 (p<0.01), whereas PTβR2I reversed the situation and significantly increased the lumen density (FIG. 5P and Q). Whether PTβR2I treatment affected the expression of angiogenic growth factors from HaCaTs, HDFs, HAECs, and THP-1-derived CD86+M1 macrophages was further investigated. It was found that the gene expressions of VEGFA and HGF in PTβR2I-treated HAECs (FIG. 5R), and PDGFBB in PTβR2I-treated M1 macrophages (FIG. 5S) were significantly enhanced. Notably, PTβR2I treatment also significantly upregulated the expression of prosurvival insulin-like growth factor 1 (IGF1) in HDFs (FIG. 5T). PTβR2I reduced the expression of inflammation cytokines from skin cells and macrophages under high glucose and TGFβ1 conditions.

Diabetes is characterized by chronic inflammation, evidenced by the increased expression of proinflammatory cytokines such as interleukin-6 (IL6), interleukin-1B (IL1B), and tumor necrosis factor-α (TNFα). Thus, whether PTβR2I modulated the expressions of inflammatory cytokines in HaCaT, HDFs, HAECs, and THP-1-derived CD86+M1 macrophages was evaluated. These cells are major sources of proinflammatory cytokines during diabetic wound healing. At the mRNA level, after PTβR2I treatment, the expressions of IL6 in M1 macrophages, of IL1B, IL6, and TNFA in HDFs, and IL1B and IL6 in HaCaTs were all significantly reduced (FIG. 5S-U).

PTβR2I Downregulated the TGFβ1/p38 Pathway without Affecting the TGFβ1/Smad2/3 Pathway Responsible for Myofibroblast Formation Under High Glucose Conditions.

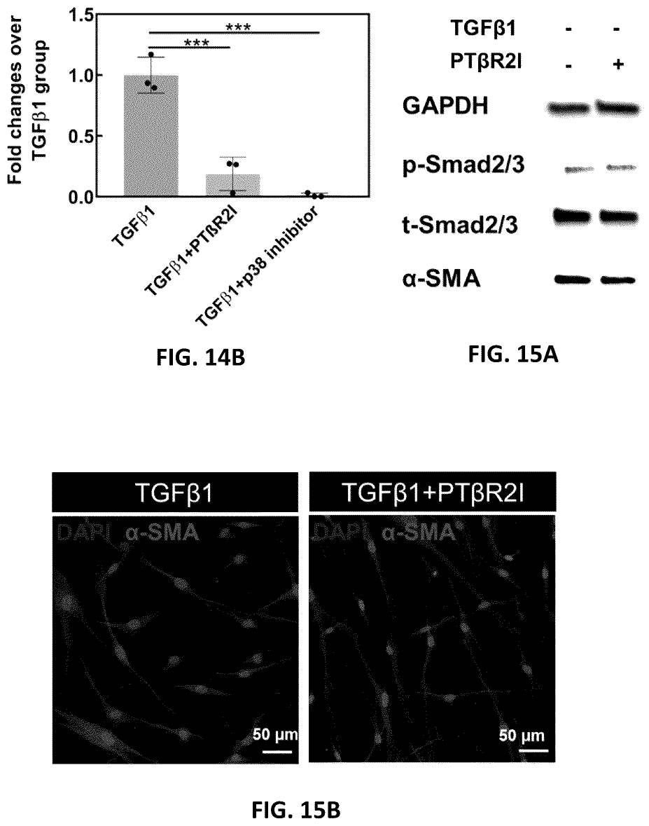

Collectively, the data demonstrate that PTβR2I treatment promoted the proliferation and migration of keratinocytes, dermal fibroblasts, and endothelial cells, stimulated endothelial morphogenesis, increased angiogenic growth factor expression in endothelial cells and macrophages, and reduced the expression of proinflammatory cytokines in keratinocytes, dermal fibroblasts, and macrophages. These actions are impaired by upregulated TGFβ1 and the high glucose condition in the diabetic wounds. To delineate the underlying mechanism, an immunoblotting study was conducted on HDFs under high glucose and TGFβ1 conditions (FIG. 5V and W). The TGFβ1/p38 pathway was targeted because it is directly associated with impaired cell migration in diabetic wounds. In addition, previous studies have shown that inhibition of TGFβ1/p38 signaling decreased inflammation during diabetic wound healing. It was observed that, relative to no TGFβ1 group, TGFβ1-treated cells displayed a nearly 4-fold increase in baseline phosphorylation of p38 (p-p38), whereas PTβR2I with TGFβ1 significantly downregulated p-p38 (p<0.05). To further confirm the role of PTβR2I in de-activating the p38 pathway, the HDFs were treated with a p-38 inhibitor (SB202190), which notably downregulated the p-p38 expression (FIG. 14).

Myofibroblasts are critical for wound healing, and the TGFβ1/p-Smad2/3 pathway represents a primary signaling pathway to drive the myofibroblast formation from fibroblasts. Whether PTβR2I treatment affected myofibroblast formation and inhibited the TGFβ1/p-Smad2/3 pathway under high glucose conditions was investigated. Interestingly, under high glucose, α-SMA-dermal fibroblasts became α-SMA+myofibroblasts in the presence of TGFβ1, regardless of the addition of PTβR2I (FIG. 15B). Consistent with these results, there was no substantial difference in the phosphorylation of Smad2/3 (p-Smad2/3) between the groups of TGFβ1 alone, and PTβR2I with TGFβ1 under high glucose condition. Similarly, the PTβR2I treatment did not alter the expression of the downstream marker α-SMA (FIG. 15A).

### Injectable, Thermosensitive, and Antioxidizing Wound Dressings can Quickly Solidify in Wounds, Significantly Improve Drug Retention in Wounds, and Sustain the Release of PTβR2I

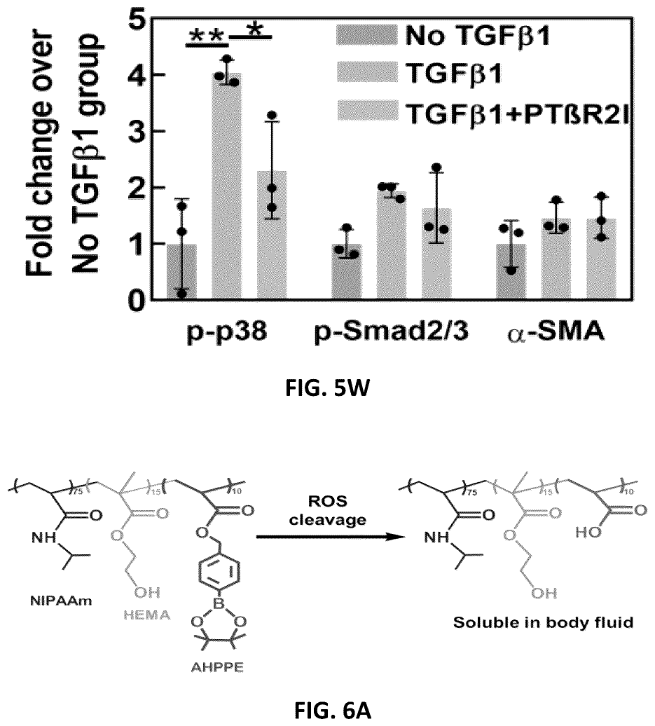

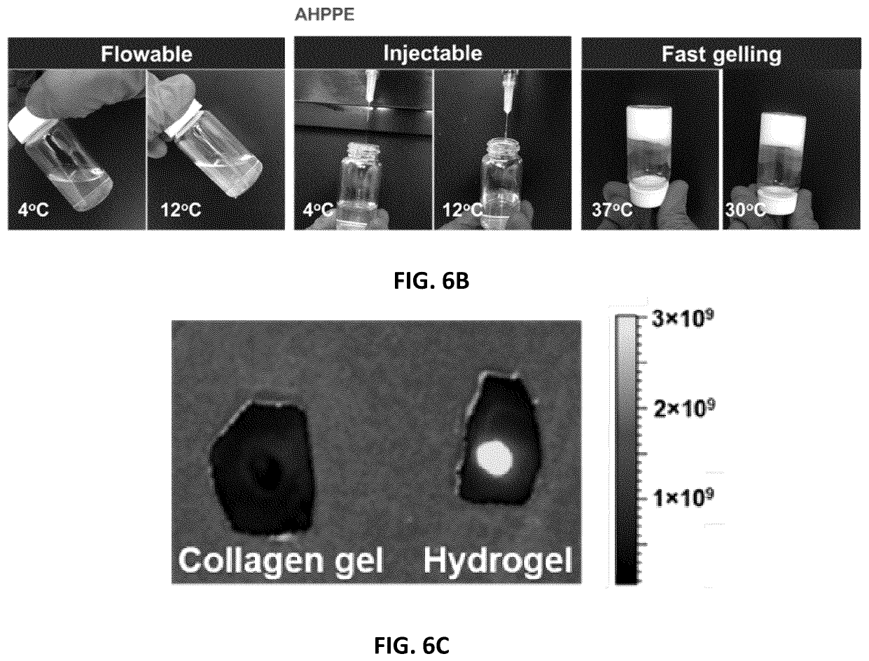

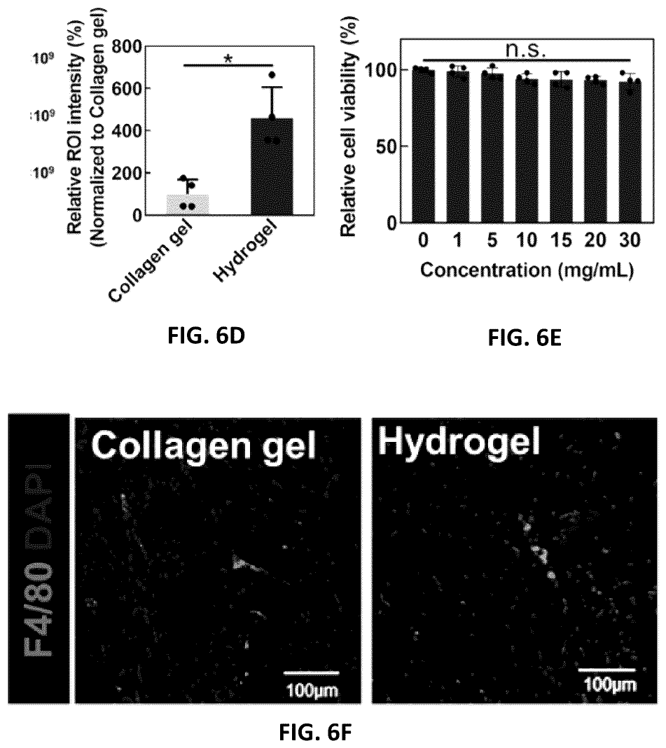

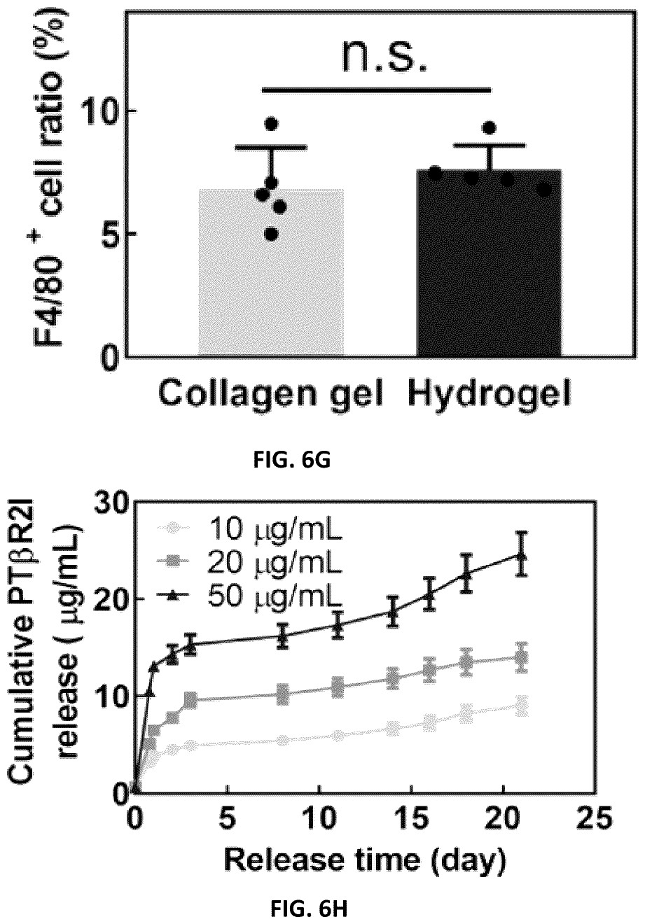

A wound dressing was further developed by encapsulating PTβR2I in an injectable, thermosensitive, and ROS-scavenging hydrogel. The hydrogel was based on N-isopropylacrylamide, 2-hydroxyethyl methacrylate, and 4-(acryloxymethyl)-phenylboronic acid pinacol ester (AHPPE) (FIG. 6A). These three components are respectively responsible for thermosensitivity, increased hydrophilicity, and ROS-scavenging. It has been demonstrated previously that a hydrogel with AHPPE effectively scavenged hydroxyl radicals and superoxide, and degraded faster in the presence of hydrogen peroxide. The hydrogel solution (6 wt %) had a thermal transition temperature of 17° C. The 4° C. and 12° C. solutions were flowable and easily injected through a 27 G needle (FIG. 6B). The 4° C. solution quickly gelled at 30° ° C.(12 s) and 37° C. (6 s). Thus, after being applied into wounds, the wound dressing solidifies rapidly to efficiently confine PTβR2I within the tissue. To confirm this action, the wound dressing was applied topically to mouse wounds. Collagen (gelation time ˜21 min) encapsulating PTβR2I was used as a control. The wound dressing solidified quickly in the tissue without any leak. In contrast, a substantial amount of collagen/PTβR2I was leaked out. When examined after 24 hours, the wound dressing had remarkably improved PTβR2I retention compared to retention by collagen/PTβR2I (FIG. 6C and D). These results show that the developed wound dressing is suitable for sustainably delivering drugs to wounds.

To evaluate the biocompatibility of the hydrogel, the final product was synthesized after the phenylboronic acid pinacol ester moiety in the hydrogel was totally removed by ROS (FIG. 6A), an MTT assay was performed on the HDFs cultured with the final product. The synthesized final product, with a thermal transition temperature of 43° C., could thus dissolve in body fluid at 37° C. HDFs cultured with or without the final product retained the same viability, even when the concentration was as high as 30 mg/ml (FIG. 6E). To further examine the hydrogel's biocompatibility, we injected the hydrogel was injected subcutaneously into the dorsal side of C57BL/6J mice. Collagen gel was used as a control group. Seven days after implantation, the tissues injected with either collagen or the hydrogel exhibited similar densities of F4/80+macrophages (FIG. 6F and G). The above results demonstrate that the hydrogel is a biocompatible wound dressing.

PTβR2I was gradually released from the wound dressing during a 21-day release study (FIG. 6H). An initial burst release was observed on the first day, followed by a slower and sustained release until day 21. The release kinetics depended on the amount of PTβR2I loaded into the wound dressings: higher loading dosages resulted in greater amounts of released PTβR2I. To determine the bioactivity of the released PTβR2I, a cell binding assay was used using HDFs treated with the PTβR2I amounts released on days 3, 8, and 14 for loading dosages of 10, 20, and 50 μg/mL. The PTβR2I released on all three time points from all the loading dosages remained bioactive (FIG. 6I).

### PTβR2I-Releasing Wound Dressings Accelerated Wound Closure in Diabetic Mice, Enhanced Keratinocyte Migration, and Promoted Hair Follicle Development

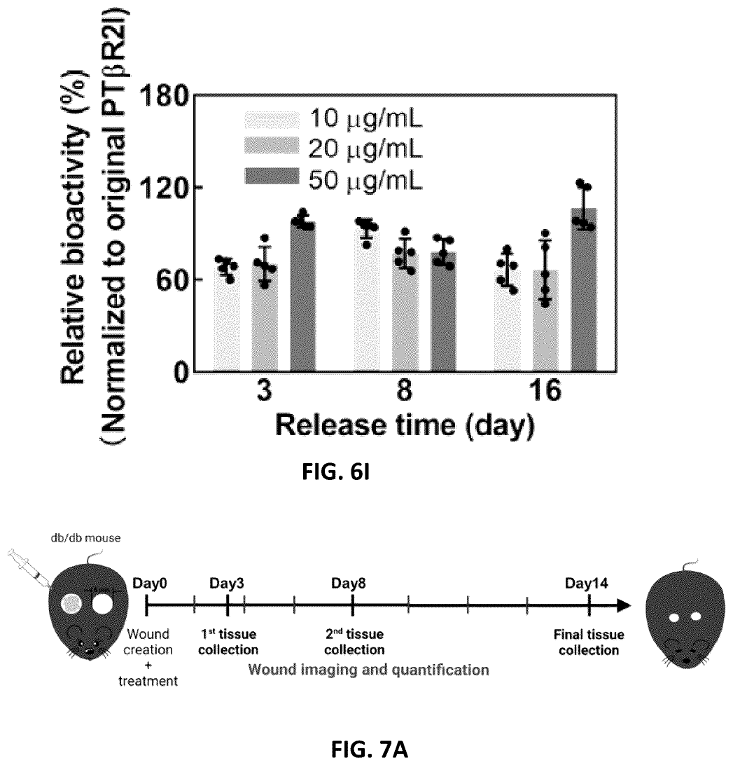

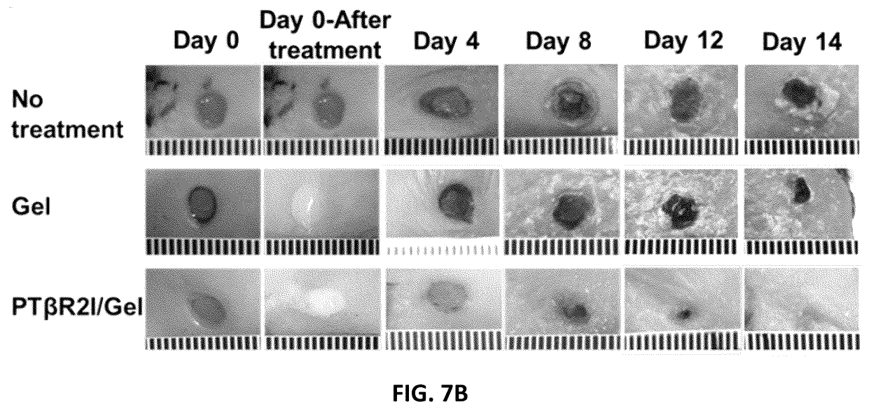

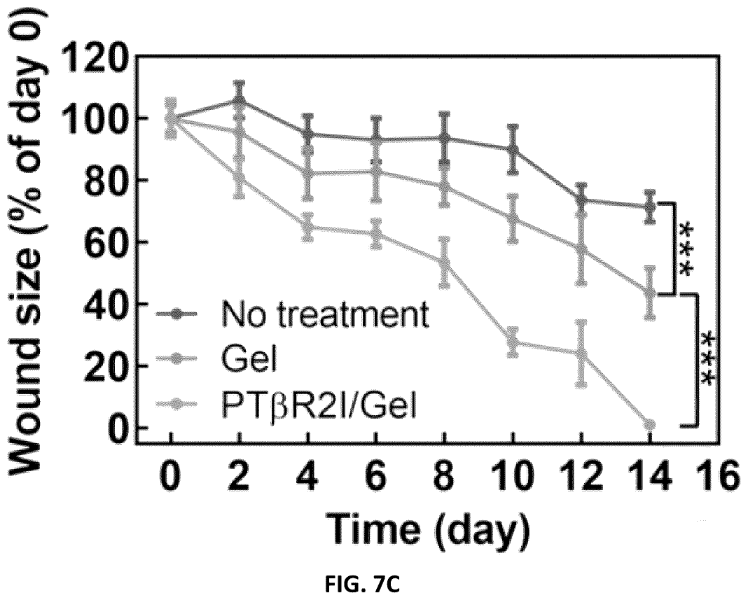

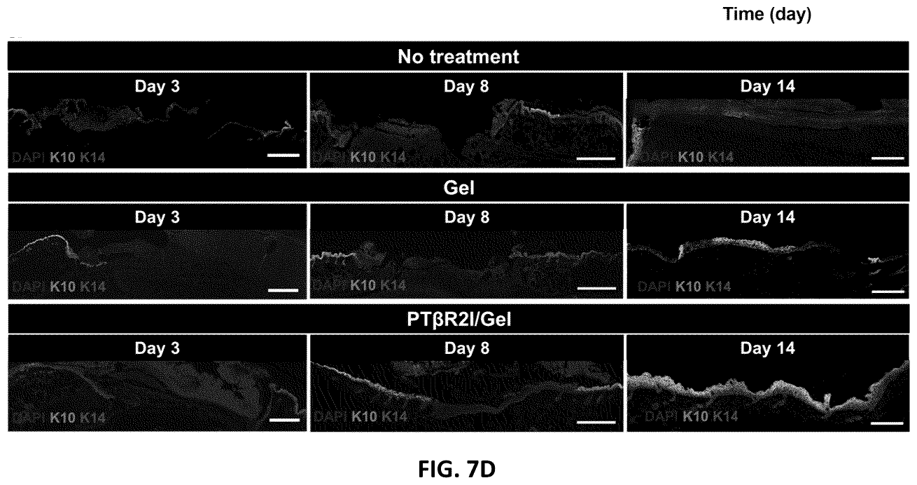

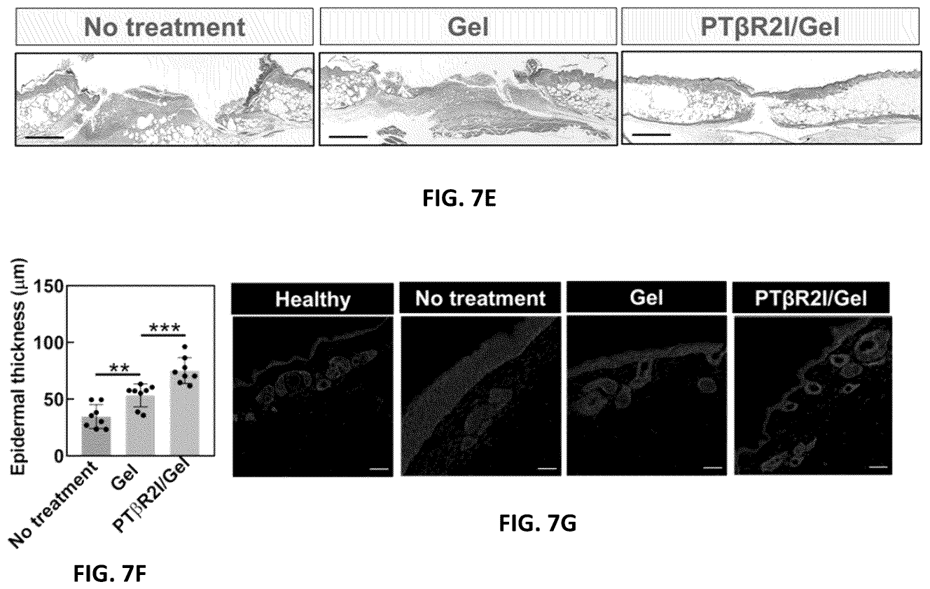

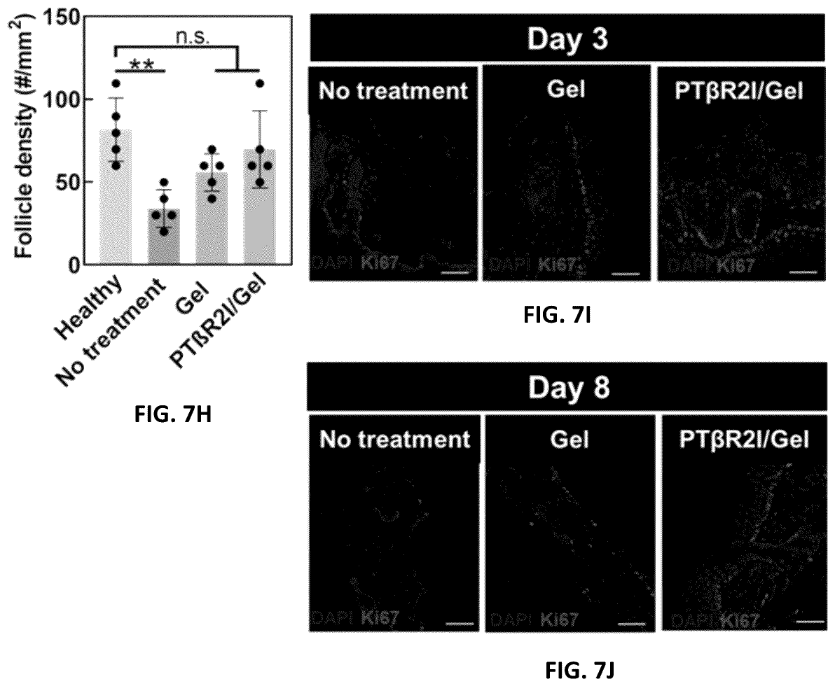

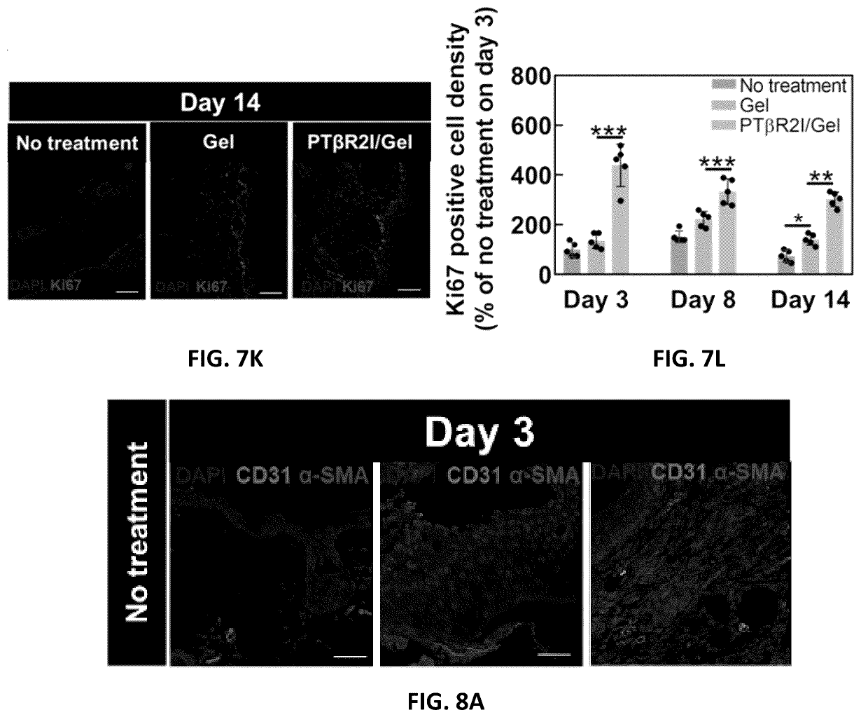

The efficacy of the PTβR2I-releasing wound dressing in accelerating wound healing was evaluated using db/db mice, a strain that represents a type II diabetes model characterized by obesity, hyperglycemia, and deficient wound closure. The wound dressings were administrated onto full-thickness excisional wounds (FIG. 7A). The wounds treated with PTβR2I-releasing wound dressing (PTβR2I/Gel group) exhibited faster wound closure than either those without treatment (No-treatment group), or those treated with wound dressing without PTβR2I (Gel group) (FIG. 7B and C). Starting from day 4, the wound size in the PTβR2I/Gel group was significantly smaller than in the No-treatment and Gel groups (p<0.05). At day 14, the wounds in the PTβR2I/Gel group were fully closed, while those in the No-treatment and Gel groups remained unclosed with wound sizes of 71.4±5.9% and 43.7±8.0%, respectively. Interestingly, treating the wounds with the wound dressings without PTβR2I (Gel group) also significantly decreased the wound size at day 14 (p<0.001) demonstrating that the hydrogel alone could accelerate wound closure.

To determine how the PTβR2I-releasing wound dressings affected the wound reepithelialization process, wounds were harvested at days 3, 8, and 14 and stained for basal keratinocytes (positive for cytokeratin 14 (K14+)), and for spinous keratinocytes (positive for cytokeratin 10 (K10+)). With increasing days of wound healing, K14+basal keratinocytes moved toward spinous layers and differentiated into K10+spinous keratinocytes80. By day 3, compared to observations of wounds dressed without PTβR2I (the Gel group) and the No-treatment group, for wounds dressed with PTβR2I (the PTβR2I/Gel group), more K14+basal keratinocytes had migrated to the wound area (FIG. 3d). By day 8, the middle stage of wound healing, the K10+ and K14+keratinocytes covered the entire wound in the PTβR2I/Gel group, while the keratinocytes had migrated more slowly in the wounds in the Gel and No-treatment groups, where a large region was without keratinocyte coverage. In addition, the PTβR2I/Gel group formed a more mature spinous layer composed of K10+keratinocytes. Consistent with the keratinocyte migration results, the wounds in the PTβR2I/Gel group exhibited the thickest epidermis on day 8 (FIG. 7E and F). The time point was determined to reflect the most significant differences in the proliferative stage of wound healing. On day 14, a visibly stratified epithelium was observed in the PTβR2I/Gel group, indicating that the reepithelialization was fully complete (FIG. 7E). In contrast, the wounds in the Gel and No-treatment groups were not fully covered by keratinocytes. Notably, larger areas of wounds in the Gel group had keratinocyte coverage than in the No-treatment group. Hair follicle development in the basal layer of the epidermis was promoted along with accelerated reepithelization. On day 14, the wounds dressed with PTβR2I/Gel exhibited the highest density of K14+hair follicles, which is a hallmark of successful healing at end stage81 (FIG. 7G and H), similar to that in surgically unwounded db/db mice (p>0.05). These results demonstrate that wound dressings based on the PTβR2I and ROS scavenging hydrogel were able to effectively accelerate keratinocyte migration and hair follicle formation, leading to quicker reepithelization of surgical wounds in diabetic mice.

The improved wound closure rate might also be the result of enhanced cell proliferation. To elucidate the relationship, the wounds were stained at days 3, 8, and 14 with a cell proliferation marker Ki67. On day 3, compared to the Gel and No-treatment groups, the PTβR2I/Gel group exhibited a remarkably greater density of Ki67+proliferating cells in both the epidermal and dermal layers (FIG. 7I. p<0.001). By day 8 and day 14 of wound healing, the density of proliferating cells had decreased in the PTβR2I/Gel group in the middle (day 8) and late (day 14) stages of wound healing, yet it remained significantly higher than in the other two groups (FIG. 7J, K, and L, p<0.001). Notably, cell proliferation was also promoted in the Gel group, where the Ki67+ cell density was greater than in the No-treatment group (p>0.05 on days 3 and 8, and p<0.05 on day 14). These results demonstrate that the PTβR2I and the ROS-scavenging hydrogel in the wound dressings can effectively stimulate cell proliferation in the epidermal and dermal layers during diabetic wound healing.

### PTβR2I-Releasing Wound Dressings Stimulated Angiogenesis and Angiogenic Growth Factor Secretion in Diabetic Wounds

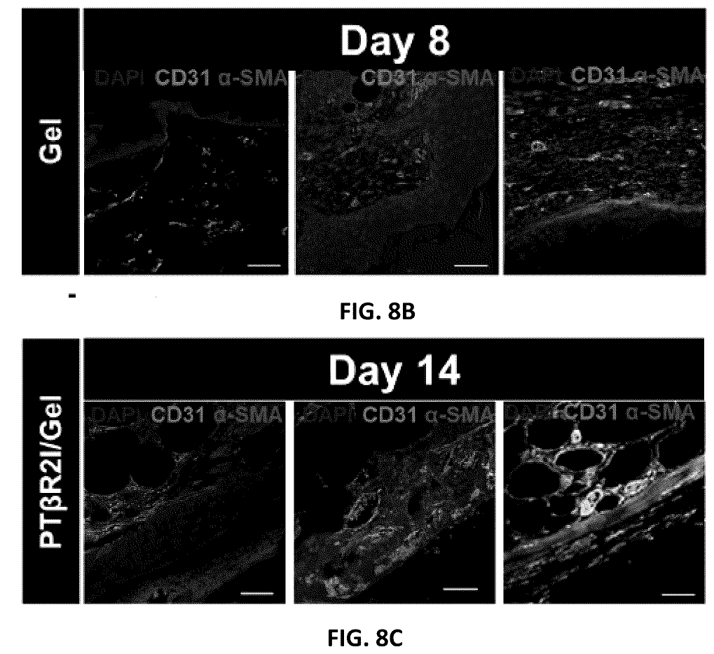

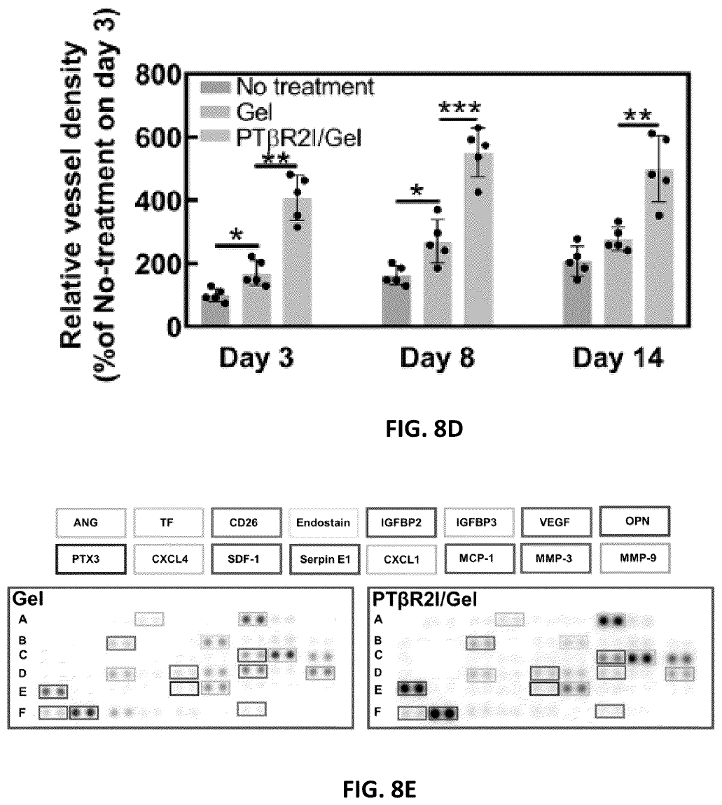

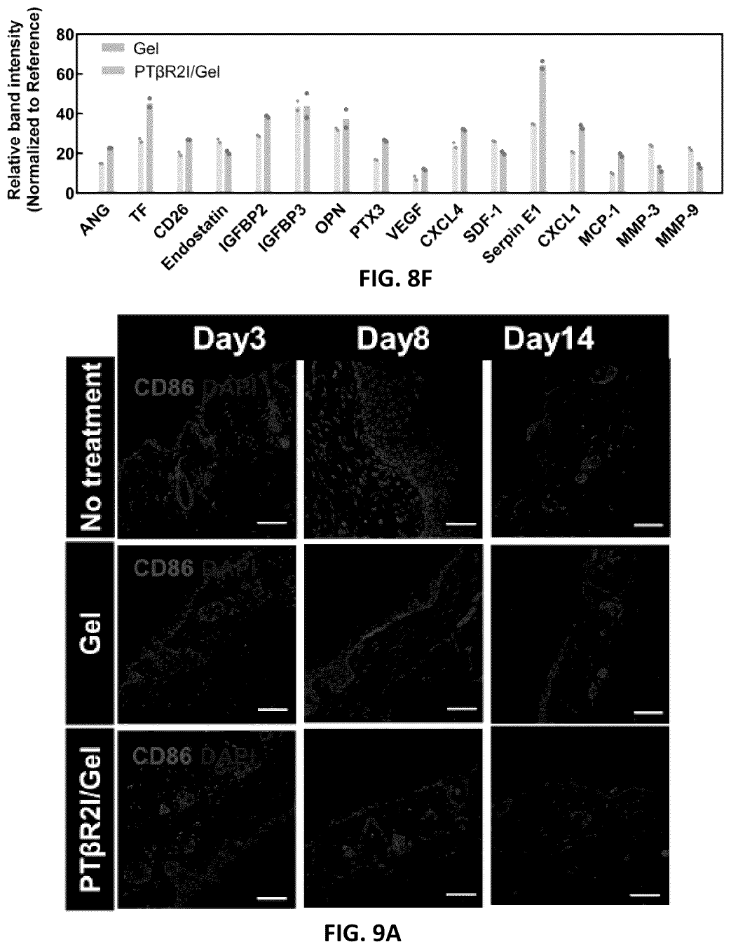

Angiogenesis is critical for healing diabetic wounds. To evaluate whether PTβR2I-releasing wound dressings stimulated angiogenesis, blood vessels in the wounds for the PTβR2I/Gel, Gel, and No-treatment groups at early (day 3), middle (day 8), and late (day 14) stages of the wound healing (FIG. 8A-D) were compared. By day 3, the group with wounds treated with gel exhibited promoted angiogenesis, with a significantly higher vessel density than the No-treatment group (p<0.05), and substantially higher vessel densities by day 8 and day 14. These observations demonstrate that the ROS-scavenging hydrogel itself was able to enhance angiogenesis. Treatment with PTβR2I/Gel wound dressing more pronouncedly stimulated angiogenesis, as evidenced by a significantly higher vessel density than in the Gel and No-treatment groups at each stage of the wound healing (p<0.01 at day 3, p<0.001 at day 8, and p<0.01 at day 14), demonstrating that the released PTβR2I can induce angiogenesis.

To further assess the role of released PTβR2I in promoting tissue angiogenesis, a protein array assay was used to examine the expression of major angiogenic factors in diabetic wounds. It was found that the PTβR2I/Gel group substantially increased the expression of angiogenic proteins, such as VEGF82,83, angiogenin (ANG)84, tissue factor (TF)85, and serpin E186 (FIG. 8E and F). These results demonstrate that PTβR2I upregulates angiogenic factor expressions in diabetic wounds.

### PTβR2I-Releasing Wound Dressings Alleviated Inflammatory Response by Decreasing M1 Macrophage Density, the Expression of Pro-Inflammatory Cytokines, and ROS Content

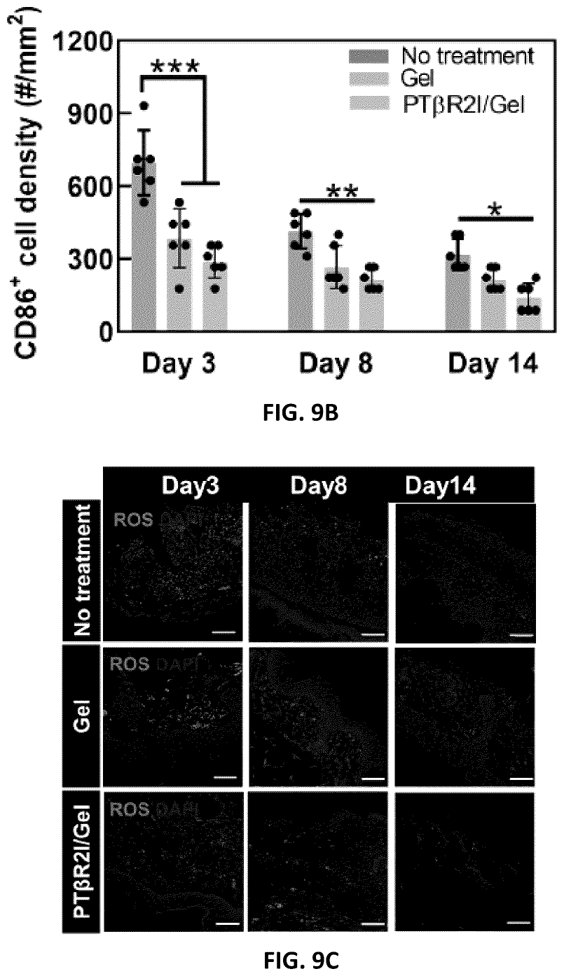

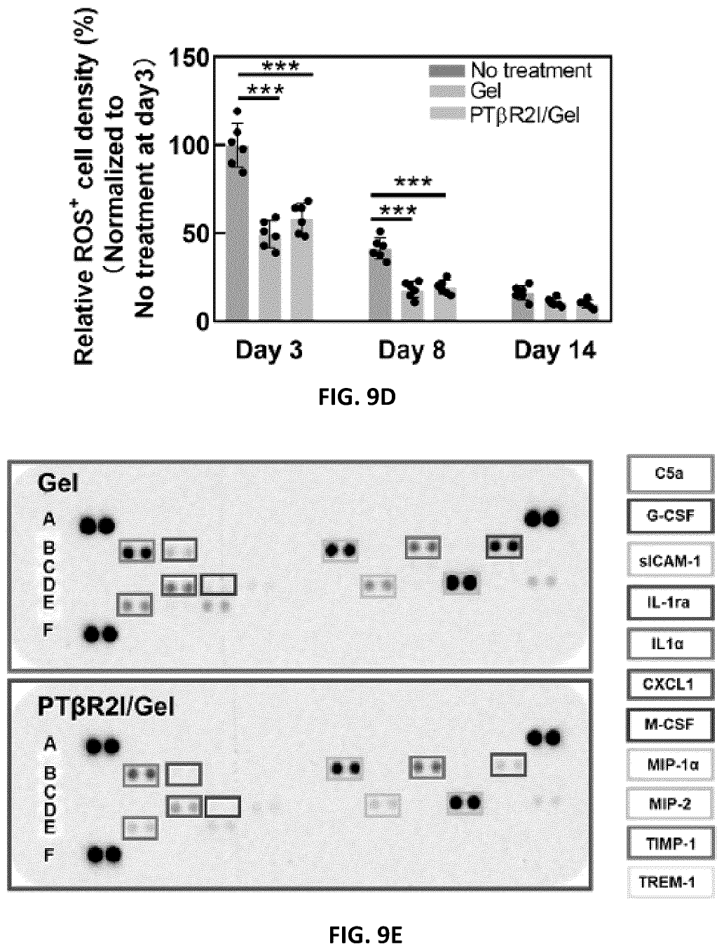

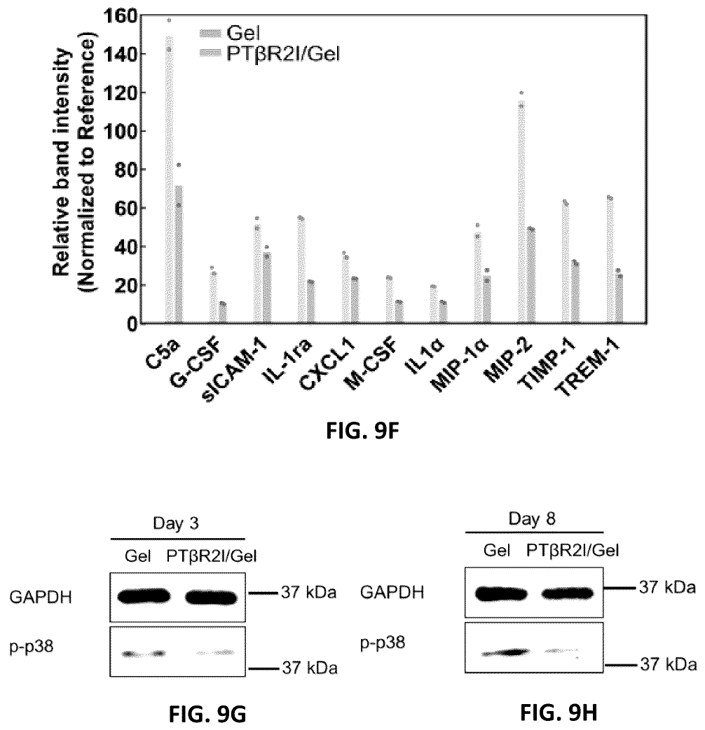

To determine whether PTβR2I-releasing wound dressings provoked an inflammatory response in diabetic wounds, the M1 macrophage density and proinflammatory cytokine expression were quantified at different stages of the wound healing (FIG. 9A and B). Compared to the no-treatment group on days 3, 8, and 14, treatment with the ROS-scavenging hydrogel alone (Gel group) substantially decreased the M1 macrophage density. Treatment with PTβR2I/Gel further reduced the M1 macrophage density observed on all three days of wound healing. In addition to decreasing the number of M1 macrophages, the PTβR2I/Gel group also remarkably decreased the expressions of various pro-inflammatory cytokines, such as complement component 5a (C5a), granulocyte colony-stimulating factor (G-CSF), interleukin-1 receptor antagonist (IL-1ra), and macrophage Inflammatory Protein-1 alpha (MIP-1a) (FIGS. 5E and 5F). These results demonstrate that PTβR2I and the ROS-scavenging hydrogel can decrease inflammation during diabetic wound healing.

As diabetic wounds heal, the overproduced ROS aggravates inflammation and delays healing. It was found that treating surgically induced wounds in diabetic mice with hydrogel (Gel group) significantly decreased the ROS content by days 3 and 8, the early and middle stages of healing (FIG. 9C and D), presumably due to the ROS scavenging property of the hydrogel. Adding PTβR2I into the hydrogel (PTβR2I/Gel group) did not change the wound ROS contents. By day 14 of the wound healing, the ROS content in both groups was reduced to an even lower level. These results suggest that the developed wound dressing can decrease oxidative stress in diabetic wound beds.

### PTβR2I-Releasing Wound Dressings Down-Regulated p38 Signaling in Diabetic Wounds

After finding that PTβR2I downregulated the TGFβ1/p38 pathway in vitro under high glucose, whether the PTβR2I released from the wound dressings into diabetic wounds also down-regulated p38 signaling was then investigated. On days 3 and 8, compared to the Gel group, the expression of p-p38 was downregulated in the PTβR2I/Gel group compared to the Gel group (FIGS. 9G and 9H). These consistent in vitro and in vivo results demonstrate that PTβR2I effectively downregulated the TGFβ1/p38 pathway, accelerating wound closure.

### PTβR2I-Releasing Wound Dressings Prevented Scar Formation in Diabetic Wounds

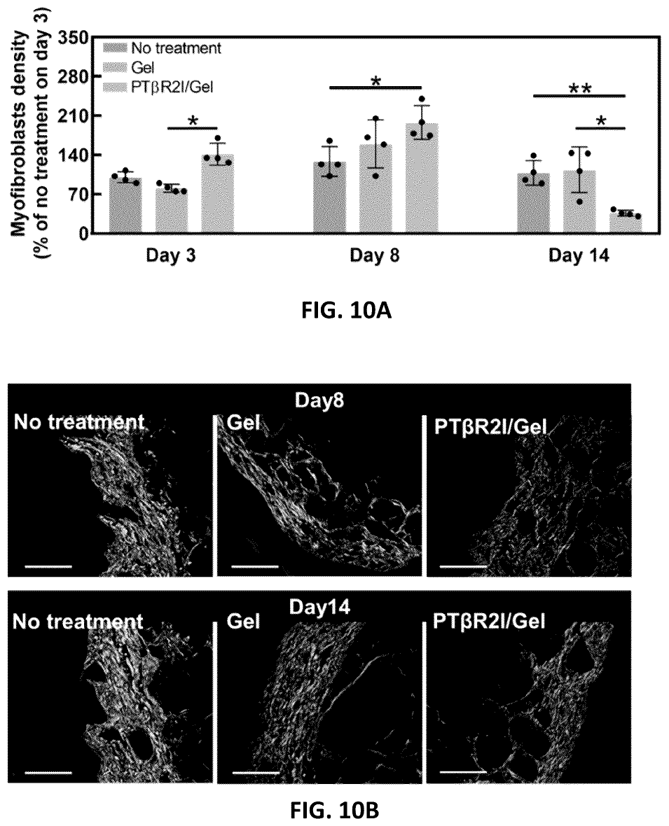

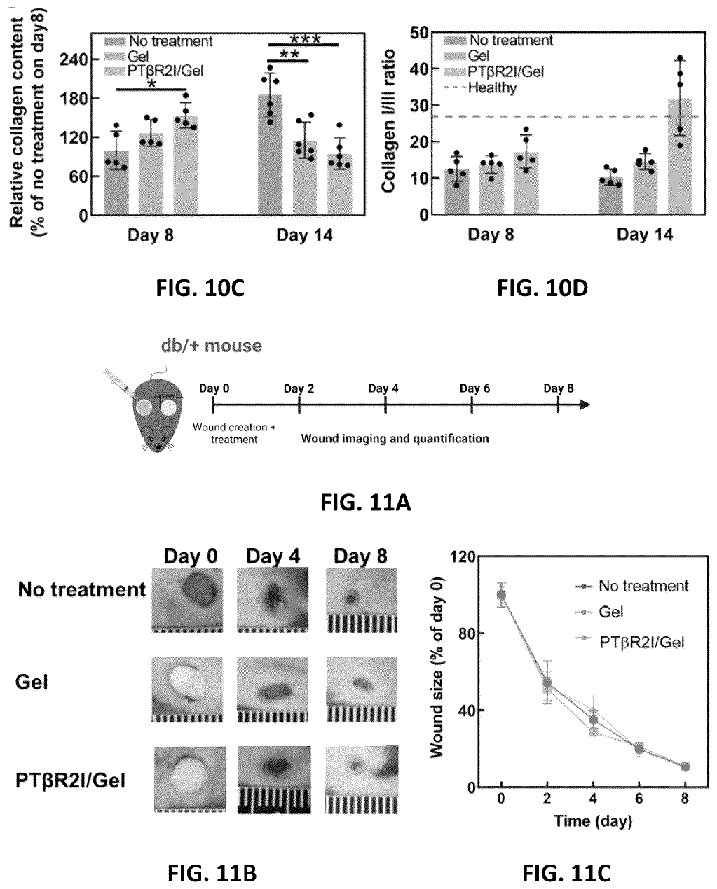

It was next sought to determine whether the accelerated reepithelization in diabetic wounds treated with PTβR2I-releasing wound dressing was associated with scar formation. Because the expression of α-SMA is also a hallmark of myofibroblasts, the myofibroblast densities in the three experimental groups on days 3, 8, and 14 of healing were quantified (FIGS. 8A, 8B, 8C, and 10A). The continuing enhanced myofibroblast density of peptide groups in the early and middle healing stages indicated fast granulation tissue formation, in which myofibroblasts are the main producer and organizer of the ECM91. The reduced myofibroblast density in the PTβR2I/Gel group on day 14 reflected nearly complete wound contraction and tissue remodeling. However, significantly more myofibroblasts were found in the No-treatment group and the Gel group in the late stage of wound healing, which may instead lead to fibrosis and abnormal scarring.

In addition, picrosirius red staining (PSR) was performed on days 8 and 14 to reveal the tissue remodeling (FIG. 10B). On day 8, the PTβR2I/Gel group showed a significantly increased collagen content compared to the Gel alone and No-treatment groups (FIG. 10C) because collagen strengthens healing tissues in the middle, proliferative stage. On the contrary, the collagen content had decreased on day 14, likely due to less collagen deposition in the remodeling process in the PTβR2I/Gel group. More specifically, as seen in FIG. 10D, PSR images were examined using a Matlab program to see how collagen type I (COLAI, yellow/red) and collagen type III (COLAIII, green) changed in different stages. Overall, for skin tissues, the most imaged collagen was type I. At day 14, both the Gel and PTβR2I/Gel groups had COLAI/III ratios that were similar to that of unwounded tissue, suggesting the completion of collagen reformation during ECM remodeling.

PTβR2I-Releasing Wound Dressings Did not Promote Wound Healing in Non-Diabetic Wounds

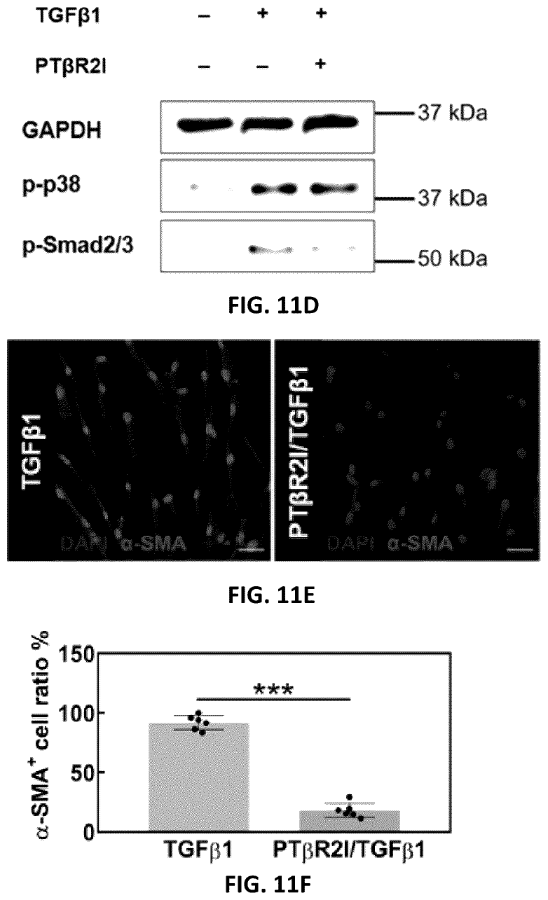

Interestingly, PTβR2I was not effective on genetically mutated db/+ mice. The same wound assay was performed on db/+ mice and monitored the wound closure for eight days (FIG. 11A). The No-treatment, Gel, and PTβR2I/Gel groups all had similar healing rates, with wound sizes less than 10% of the original after 8 days (FIG. 11B and C). To delineate the mechanism, in vitro immunoblotting was performed on dermal fibroblasts under normal glucose conditions that mimicked the microenvironment of db/+ mice (FIG. 11D). PTβR2I apparently inhibited the TGFβ1/p-Smad2/3 pathway, but did not downregulate p-p38, aligning with in vivo results. The IF study of myofibroblast activation further validated this result (FIGS. 11E and 11F).

Overall, although PTβR2I showed an apparent inhibition of TGFβ1/p-Smad2/3 at normal glucose levels, no effect was observed when the glucose level was as high as found in diabetes. Instead, PTβR2I modulated the TGFβ1/p38 pathway under high glucose, and the downregulation of p-p38 led to accelerated wound healing in db/db mice.

### DISCUSSION

Impaired wound healing is a lifetime risk for patients with diabetes. Various therapies have been developed to heal diabetic wounds, yet effective treatment remains a challenge, largely because current therapies cannot efficiently address the key intrinsic causes of slow diabetic wound healing, i.e., abnormal skin cell functions (particularly migration), delayed angiogenesis, and chronic inflammation. TGFβ1/p38 signaling is directly associated with these key intrinsic causes. The p-p38 is upregulated in the wounds of db/db mice with sustained hyperglycemia. Using TGFβ inhibitors or anti-TGFβ antibodies to decrease the amount of active TGFβ1 in wounds can reduce its deteriorating effect. However, it is challenging to inhibit only the TGFβ1/p38 pathway and not interfere with the TGFβ1 signaling axes that are essential for wound healing, such as the TGFβ1/Smad2/3 pathway. Therefore, wound dressings that selectively regulate TGFβ1 signaling under hyperglycemia can potentially promote diabetic wound healing.

The experimental results described above demonstrated that wound dressings consisting of PTβR2I and a ROS-scavenging hydrogel accelerated wound healing in db/db mice. It was demonstrated that PTβR2I acts by binding with TβR2 and differentially regulating the TGFβ1 pathways, i.e., down-regulating the TGFβ1/p38 pathway while not affecting the TGFβ1/Smad-2/3 pathway under the hyperglycemia characteristic of diabetes. It was found that PTβR2I enhanced the migration of keratinocytes, dermal fibroblasts, and endothelial cells; promoted cell proliferation and paracrine effects; facilitated endothelial morphogenesis; and decreased proinflammatory cytokine expression in skin cells under TGFβ1 and high glucose conditions (FIG. 5J-T). An injectable, thermosensitive, fast-gelling, and ROS-scavenging hydrogel was used to develop a PTβR2I-releasing wound dressing. The thermosensitivity and fast-gelling properties allowed the PTβR2I to be highly retained in the wounds after administration (FIG. 6C). The ROS-scavenging property enabled the wound dressing to capture the up-regulated ROS in diabetic wounds (FIGS. 6A and 9C and D). The PTβR2I-releasing wound dressing significantly accelerated reepithelization, promoted host cell proliferation and vessel formation, decreased inflammation and oxidative stress, and regulated collagen deposition without forming scar tissue. Intriguingly, under euglycemia, PTβR2I inhibited the TGFβ1/Smad-2/3 pathway without affecting the TGFβ1/p38 pathway, resulting in no improvement of wound healing when tested in db/+ mice (FIG. 11D).

Keratinocytes, fibroblasts, and endothelial cells are respectively responsible for epithelialization, wound contraction, and angiogenesis during wound healing. In diabetic wounds, the TGFβ1 and high glucose conditions impair the migration of these cells, leading to delayed epithelialization, wound contraction, and angiogenesis. In contrast, it was found that PTβR2I enhanced the migration of keratinocytes, fibroblasts, and endothelial cells under TGFβ1 and high glucose conditions (FIG. 5M-O). Following cell recruitment, cell proliferation also plays an important role in wound healing, especially for larger and chronic diabetic wounds where migration alone is insufficient for closure. It was demonstrated that treatment with PTβR2I increased the proliferation of keratinocytes, fibroblasts, and endothelial cells. Notably, PTβR2I also stimulated endothelial cells to form lumens (FIGS. 5P and 5Q) and upregulated the expressions of VEGFA and HGF in endothelial cells (FIG. 5R). These factors not only promote angiogenesis but also positively affect the migration, proliferation, and differentiation of keratinocytes. As a result, the diabetic wounds treated with PTβR2I-releasing wound dressings had significantly greater vessel density than those dressed without PTβR2I (FIG. 8A-D). The PTβR2I released from the wound dressings favorably influenced the migration of keratinocytes throughout the inflammatory, proliferative, and remodeling phases of wound healing (FIG. 7D). In addition, more K14+keratinocytes in the basal layer underwent differentiation towards K10+keratinocytes in the suprabasal layers.

Chronic inflammation and high oxidative stress critically delay diabetic wound healing. It was demonstrated that PTβR2I suppressed the expression of proinflammatory cytokines such as TNFα, IL1B, and IL6 in keratinocytes, fibroblasts, and macrophages in vitro (FIG. 5S-U). This result is consistent with previous reports that inhibiting the p38 pathway reduced the expression of pro-inflammatory cytokines in skin cells. In diabetic wounds, PTβR2I released from the wound dressings decreased the M1 macrophage density at all three stages of the healing (FIG. 8A and B). Overproduced ROS in diabetic wounds can contribute to chronic inflammation. It was shown that ROS-scavenging hydrogel in the wound dressing can scavenge ROS and continuously mitigate the oxidative stress.

Diabetic wounds are characterized by impaired production of ECM, a crucial facilitator of wound healing, from the inflammatory phase through the remodeling phase. Collagen, the most abundant ECM in the skin, is significantly underproduced in diabetic wounds. In addition, the ratio of the two major collagen types (I and III) is abnormally different from that of uninjured skin. Myofibroblasts are primarily responsible for collagen deposition in wounds. During normal wound healing, the number of myofibroblasts decreases dynamically in the final remodeling stage to avoid excessive collagen deposition that causes scars. For these reasons, it is important to regulate myofibroblasts to achieve normal deposition of collagen and a normal collagen I/III ratio. This Example demonstrated that PTβR2I-releasing wound dressings beneficially modulated the number of myofibroblasts in the progression from the inflammatory phase to the remodeling phase (FIG. 10A), most probably because PTβR2I did not interfere with the TGFβ1/Smad2/3 pathway. Collagen deposition was increased during the inflammatory and proliferative phases and decreased during the remodeling phase without inducing scar formation. In addition, the collagen I/III ratio in the remodeling phase was similar to that of uninjured tissue.

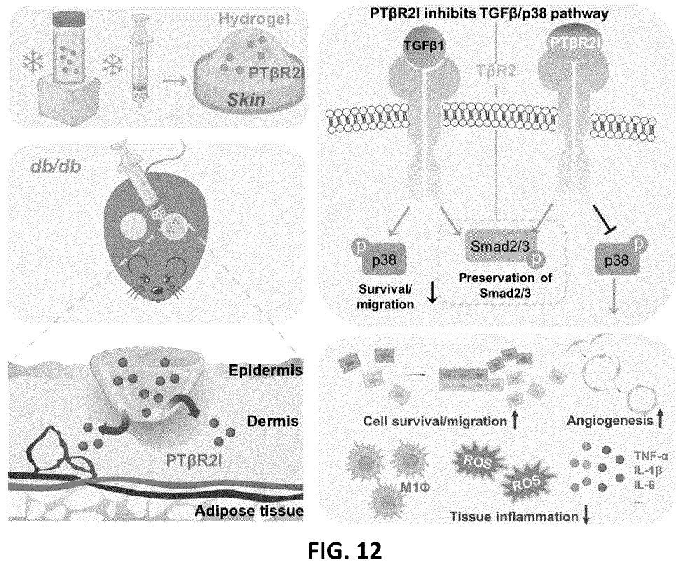

In summation, it is reported herein that wound dressings consisting of PTβR2I and a ROS-scavenging hydrogel accelerated diabetic wound healing, by differentially regulating the TGFβ1/p38 and the TGFβ1/Smad2/3 pathways (FIG. 12). The wound dressings performed multiple functions: stimulating skin cell migration, proliferation and paracrine effects; promoting endothelial morphogenesis and angiogenesis; and reducing tissue inflammation and oxidative stress under TGFβ1 and high glucose conditions. These wound dressings more quickly accelerated wound closure than using growth factors, protein, exogenous cells, or oxygen therapy in the same animal model. In future studies, we will test the developed wound dressings in large animals (e.g., pigs with and without diabetes), or in models with concomitant hyperlipidemia and vascular occlusive disease.

### Methods

### Materials

All chemicals were purchased from Millipore-Sigma unless otherwise stated. N-isopropylacrylamide (NIPAAm, TCI) was recrystallized three times before use. Hydroxyethyl methacrylate (HEMA, Alfa Aesar) was used after passing it through an inhibitor removal column. Acryloyl chloride, 4-(hydroxymethyl)-phenylboronic acid pinacol ester (HPPE), acrylic acid (AAc), and benzoyl peroxide (BPO, Life Technologies) were used as received.

### Binding of PTβR2I to TBR2, TβR1 and IgG

To test the ability of PTβR2I to bind to TβR2, a binding assay was performed. TβR1, TβR3, and IgG were used as controls. In brief, ELISA plates (Costar) were coated with 20 nM of TβR2 (R&D), TβR1 (R&D), or IgG (R&D) and incubated at 4° C. overnight. The plate was blocked with Tris-buffered Saline (TBS) containing 5% bovine serum albumin (BSA) for 1 hour. Then PTβR2I (0.5 μg/mL) was added to the plate and incubated for 2 hours. The fluorescence intensity was read by a microplate reader (Molecular Devices) with a predefined wavelength (excitation/emission=485/535 nm).

To test PTβR2I binding to cells, HDFs (Lonza) were seeded on type I collagen-coated cover glasses and inserted in a 6-well plate. After 24 hours, the following treatments were added into the wells: 1) PTβR2I solution (10 μg/mL) for 48 hours; 2) PTβR2I solution (10 μg/mL) for 24 hours, and then TGFβ1 (10 ng/ml) for another 24 hours; 3) TGFβ1 (10 ng/mL) for 24 hours, and then PTβR2I (10 μg/mL) for another 24 hours; and 4) both PTβR2I (10 μg/mL) and TGFβ1 (10 ng/ml) for 48 hours. Control wells contained HDF-seeded cover glasses without added PTβR2I or TGFβ1, and incubated for 48 hours. After the treatment, cells were fixed by 4% paraformaldehyde and blocked with 10% goat serum in Dulbecco's phosphate-buffered saline (DPBS). Primary antibody mouse anti-TβR2 (1:300, Santa Cruz) that was diluted in DPBS containing 3% BSA was incubated with the cells overnight at 4° C. The cells were washed three times with DPBS. A secondary antibody Alexa Fluro-647 (1:300, Fisher Scientific) was then applied for 2 h at room temperature, followed by incubating with DAPI (Millipore-Sigma) for five minutes.

To validate the above results, the fluorescence intensity of PTβR2I bound to the HDFs was measured for each treatment. Briefly, HDFs (Lonza) were seeded on type I collagen-coated 96-well black microplates. After 24 hours, the treatments described above were applied. Three wells on the plate were washed three times with DPBS to remove free PTβR2I. The fluorescence intensity was measured using the same microplate reader.

### Cell Culture

HDFs were passaged using Gibco Dulbecco's modified Eagle's medium (DMEM, Gibco) supplemented with 10% fetal bovine serum (FBS, Atlanta Biologicals) and 1% penicillin-streptomycin (P/S). Human keratinocytes HaCaT cells were purchased from AddexBio and cultured in AddexBio-Optimized DMEM containing 10% FBS and 1% P/S. Human arterial endothelial cells (HAEC) were obtained from Cell Systems and cultured in a complete medium kit supplemented with serum (FBS, 10%), and culture boost. Each of the three cell types was incubated at 37° C. with 5% CO2 until reaching 80-90% confluency before passaging. The culture medium was replenished every other day.

Macrophages were derived from THP-1, a human monocytic cell line (ATCC). THP-1 cells were treated with 100 ng/ml phorbol 12-myristate 13-acetate (PMA, Millipore-Sigma, P1585) for 48 hours, followed by 24 hours in RPMI 1640 medium (ATCC) with 10% FBS and 1% P/S. To induce differentiation into M1 macrophages, the cells were treated with 100 pg/mL lipopolysaccharide (LPS, Millipore-Sigma) and 20 ng/ml interferon-gamma (IFN-γ, Millipore-Sigma) for 48 hours.

### Cell Proliferation Under High Glucose and TGFβ1 Conditions

HDFs, HaCaTs, and HAECs were seeded in 96-well plates at respective densities of 1.5×10⁴ cells/mL, 2×10⁴ cells/mL, and 2×10⁴ cells/mL. High glucose (4.5 g/L) basal medium with 1% P/S was used for the culture. After 24 hours, TGFβ1 (10 ng/ml) or TGFβ1 (10 ng/ml)/PTβR2I (10 μg/mL) was added to the medium. The culture continued for three days. The medium was then replaced by papain solution and incubated at 60° C. for 24 hours. The dsDNA content was tested using a Quant-iT™ PicoGreen dsDNA Assay Kit (Invitrogen).

### Cell Migration Under High Glucose and TGFβ1 Conditions

HDFs, HaCaTs, and HAECs were cultured in 6-well plates using serum-free high glucose (4.5 g/L) basal medium with 1% P/S. After the cells reached confluency, a 200-μL pipet tip was used to scrape the cell monolayer. Then 3 mL of medium containing TGFβ1 (10 ng/ml) or TGFβ1 (10 ng/ml)/PTβR2I (10 μg/mL) was added to the wells. The cells were imaged at predetermined time points using a bright-field microscope. The distances between the scratch walls were measured using ImageJ to calculate the migration ratio

### Endothelial Cell Lumen Formation Under High Glucose and TGFβ1 Conditions

A 3D collagen gel model was used to evaluate endothelial cell lumen formation under high glucose and TGFβ1 conditions. Briefly, the collagen gel was formed by mixing 4 mg/ml of rat tail type I collagen solution (Life Technologies), FBS, DMEM, and NaOH. Then 500 μL of the mixture was transferred to a 48-well plate and placed in a 37° C. incubator for 30 minutes to allow gelation. HAECs were then seeded into the collagen gel at a density of 2×10⁴ cells/well. TGFβ1 (10 ng/ml) or TGFβ1 (10 ng/ml)/PTβR2I (10 μg/mL) was then added. After three days of culturing, cells were stained with F-actin (Abcam, ab176753) and DAPI (Millipore-Sigma). The constructs were imaged by

Olympus FV1200 confocal microscopy with z-stack mode. The lumen density was calculated from the images.

mRNA Expression for In Vitro Cultured Cells

To determine the mRNA expression of the cells (HDFs, HaCaTs, and HAECs) treated with TGFβ1 (10 ng/ml) or TGFβ1 (10 ng/ml)/PTβR2I (10 μg/mL) under high glucose conditions, RNA was extracted using Trizol (Invitrogen) and reverse transcribed using a cDNA synthesis kit (Applied Biosystems). Gene expression was performed by real-time RT-PCR, using Maxima SYBR Green/Fluorescein Master Mix (Thermofisher) and selected primer pairs, shown in Table 1 below. β-actin served as the housekeeping gene. The ΔΔCt method was used for data analysis.

TABLE 1
RT-PCR Primer Sequences

| Gene | Forward (5′-3′) | Reverse (5′-3′) |
|---|---|---|
| IL1B | CTAAACAGATGAAGTGCTCC | GGTCATTCTCCTGGAAGG |
| | (SEQ ID NO. 2) | (SEQ ID NO. 3) |
| IL6 | GCAGAAAAAGGCAAAGAATC | CTACATTTGCCGAAGAGC |
| | (SEQ ID NO. 4) | (SEQ ID NO. 5) |
| PDGFBB | GGGCAGGGTTATTTAATATGG | AATCAGGCATCGAGACAG |
| | (SEQ ID NO. 6) | (SEQ ID NO. 7) |
| VEGFA | AATGTGAATGCAGACCAAAG | GACTTATACCGGGATTTCTTG |
| | (SEQ ID NO. 8) | (SEQ ID NO. 9) |
| HGF | CAAGGACCTACGAGAAAATTAC | ATCACAGTTTGGAATTTGGG |
| | (SEQ ID NO. 10) | (SEQ ID NO. 11) |
| IGF1 | CCCAGAAGGAAGTACATTTG | GTTTAACAGGTAACTCGTGC |
| | (SEQ ID NO. 12) | (SEQ ID NO. 13) |

### Protein Array Assay

After three days post-wound assay on db/db mice, the wounded tissues were collected and lysed. The protein concentrations were quantified by Bradford assay. The samples were tested using a Proteome Profiler Mouse Angiogenesis Array kit (R&D Systems) and a Mouse Cytokine Array kit (R&D Systems). The intensities of the dots on the membranes in the kits were quantified using Image Lab software.

### Synthesis and Characterization of Hydrogel

The ROS-responsive monomer 4-(acryloxymethyl)-phenylboronic acid pinacol ester (AHPPE) was synthesized by acrylation of HPPE following a previously established method. The hydrogel was synthesized by free radical polymerization of N-isopropylacrylamide (NIPAAm), hydroxyethyl methacrylate (HEMA), and AHPPE using benzoic benzoyl peroxide (BPO) as an initiator. The molar feed ratio of the three monomers was 75/15/10. The chemical structure of the synthesized hydrogel was confirmed by 1 H-NMR. After AHPPE was completely cleaved by ROS, the final product poly (NIPAAm-co-HEMA-co-acrylic acid(AAc)) was synthesized using the same approach. The molar feed ratio of NIPAAm/HEMA/AAc was 75/15/10. The hydrogel solution of 6 wt. % was prepared by dissolving the polymer into DPBS with continuous stirring at 4° C. for 12 hours. The solution was then kept on ice until use.

The injectability of the hydrogel solution was examined at 4° C. and 12° C., using a 27G needle. The gelation time of the 4° C. solution was evaluated at 30° C. and 37° C. To test the cytotoxicity of the degradation product, the final product (FIG. 7A) was dissolved in DMEM with 10% FBS at different concentrations. HDFs were seeded at a density of 3×10⁴ cells/mL in a 96-well plate. After 24 hours, the cells were treated with the medium containing the degradation product, and after 48 hours, the cell viability was quantified by MTT assay. The medium without degradation product was used as a control.

### Hydrogel and PTβR2I Retention in Diabetic Wounds

To evaluate the retention of hydrogel and PTβR2I in diabetic wounds, PTβR2I (FITC-labeled) was mixed separately with the hydrogel solution and the collagen solution. The collagen solution was used as a control. All animal experiments were performed in accordance with the National Institutes of Health Guide for the Care and Use of Laboratory Animals. The protocol was approved by the Institutional Animal Care and Use Committee of Washington University in St. Louis. Female BKS.Cg-Dock7m+/+Leprdb/J mice (db/db mice, Jackson Laboratories) aged eight weeks were used. The mice were anesthetized by isoflurane inhalation, and an electronic shaver and hair removal cream were used to thoroughly remove the hair from the dorsal skin. Before surgery, ethanol pads and betadine were applied in series on the dorsal skin. Then, with a biopsy punch, two symmetric 5-mm diameter wounds were made on the dorsal skin of each mouse. Each wound was then dressed with either the PTβR2I/hydrogel mixture or the PTβR2I/collagen mixture. After 24 hours, the mice were sacrificed and the wounds were collected, followed by imaging using an In Vivo Imaging System (IVIS Spectrum, Perkin Elmer Inc, Waltham, MA, USA) with the excitation filter of 485 nm and an emission filter of 535 nm. The fluorescence images were quantified by Living Image software (PerkinElmer Inc.).

### Subcutaneous Implantation of Hydrogel

To examine the in vivo toxicity of the hydrogel, 6 wt. % hydrogel solution was subcutaneously injected into the 8-week-old C57BL/6J mice. Prior to the injection, the pre-cooled hydrogel solution was sterilized under UV light for 30 minutes. As controls, mice injected with collagen gel were used. After seven days, tissue specimens were harvested at the injection sites and fixed with 4% paraformaldehyde for 24 hours. The tissue sections (5 μm thick) were stained with anti-F4/80 antibody (Santa Cruz) and DAPI, respectively. The stained sections were imaged with an Olympus FV1200 confocal microscope, and the F4/80 cell ratio was quantified from the images.

Fabrication of Wound Dressings and Kinetics of PTβR2I Release from Wound Dressings

Wound dressings were fabricated by encapsulating PTβR2I in the hydrogel solution at 4° C. Briefly, PTβR2I solution was mixed with 6 wt. % hydrogel solution to reach final PTβR2I concentrations of 10 μg/mL, 20 μg/mL and 50 μg/mL. The mixtures were stirred at 4° C. for 12 hours. To determine PTβR2I release kinetics, the mixtures were first incubated at 37° C. to gel. After 1 hour, the supernatant was replaced by a release medium (DPBS with 1% P/S). The medium was then collected at pre-determined time points for 21 days, and a fresh release medium was added after each collection. The concentration of the released PTβR2I was determined by measuring the fluorescence intensity and calculating the corresponding concentration using a standard curve. The bioactivity of the released PTβR2I was evaluated by its ability to bind to the TβR2 on the HDFs. The collected released medium was added to the culture medium of HDFs that were seeded in a 96-well plate. After 2 hours of incubation, the cells were washed three times with DPBS, and the fluorescence intensity was measured using a microplate reader (excitation/emission=485/535 nm).

Implantation of Wound Dressings into Diabetic Wounds

Eight-week-old, female db/db mice were used. Carprofen tablets (1/4 tablet per mouse) were given orally 48 hours before surgery and continued for seven days after the surgery as an analgesic. One day before surgery, the blood glucose levels of the mice were tested with a glucometer to confirm they were above 300 mg/dL. The mice were anesthetized by isoflurane inhalation, and the dorsal surface was thoroughly shaved. Then a 5-mm diameter biopsy punch (VWR) was used to create two symmetric full-thickness wounds on the back of each mouse. Next, 100 μL of liquid wound dressing, with or without 50 μg/mL of PTβR2I, was applied topically to the wound sites. The mice were observed every other day, and digital photographs of the wounds, with a ruler at the side, were acquired by a digital camera. Wound sizes were calculated using Image J.

### Western Blotting Assay for In Vitro Cultured Cells and Diabetic Wounds

For in vitro tests, protein lysates were collected from HDFs. Pre-cooled cell scrapers were used to detach the cells, and the cells were re-suspended in RIPA lysis buffer with the inhibitor cocktail. For in vivo tests, wound skin tissues were washed three times with pre-cooled DPBS, dissected into small pieces, and re-suspended in RIPA lysis buffer containing protease and phosphatase inhibitors. Homogenization was then performed thereafter with an ultrasonic processor (Cole Parmer). After centrifugation at 12,000 g for 20 min, the supernatants were collected, and protein concentrations were measured using a Bradford protein assay kit (Bio-rad).

The protein samples were separated by 10% Mini-PROTEAN TGX stain-free precast gels (Bio-rad) and transferred onto an immune-blot PVDF membrane. The blots were washed three times with DPBS containing 0.1% Tween 20 (PBST), blocked with 5% milk powder in PBST buffer for 40 min, and incubated with primary antibodies at specific dilutions overnight at 4° C. The primary antibodies included rabbit GAPDH antibody (1:5000, Cell signaling), rabbit anti-a-smooth muscle actin (α-SMA) antibody (1:2000, Cell signaling), rabbit anti-phospho-Smad2(Ser465/467)/Smad3 (Ser423/425) (1:500, Cell signaling), and rabbit anti-phospho-p38 (1:500, Cell signaling). The membranes were washed three times with PBST and incubated with horseradish peroxidase (HRP)-conjugated secondary antibodies (1:2500). Immunoblots were then washed with PBST and detected with a WesternBright HRP substrate detection kit (Advansta). The ChemiDoc XPS+system (Bio-rad) was used to image the blots.

### Histological and Immunofluorescence Analyses

Wound tissues were collected on days 3, 8, and 14 after administration of wound dressings. The tissues were fixed in 4% paraformaldehyde for 24 hours. Then the samples with the whole wound areas were cross-sectioned into 5 μm thick slices, and Masson's Trichrome staining (MTS), and Picosirius red staining were performed. The epidermal thickness was calculated from the MTS images in the wounded region. For immunohistochemical staining, tissue sections were stained with primary antibodies including mouse anti-cytokeratin 14 (abcam) antibody, rabbit anti-cytokeratin 10 (abcam), rabbit anti-CD31 (abcam), mouse anti-α-SMA (abcam) and rat anti-Ki67 (Thermofisher), and incubated at 4° C. overnight. Alexa 647 goat anti-rabbit, Alexa 546 goat anti-mouse, and Alexa 488 goat anti-rabbit secondary antibodies (Thermofisher) were then applied. DAPI (Millipore-Sigma) was used to stain the nuclei. Images were acquired by Olympus confocal microscope and analyzed using ImageJ.

### Statistical Analysis and Reproducibility

Data are presented as mean±standard deviation. Statistical analysis was performed using one-way or two-way ANOVA followed by the Bonferroni post-test, using GraphPad Prism 8. Significance was defined as P<0.05.

What is claimed is:

1. A method of promoting healing of a wound in a diabetic patient, comprising applying a hydrogel loaded with a therapeutically effective dosage of a TGFβ receptor II (TGFβRII) inhibitor to the wound.

2. The method of claim 1, wherein the TGFβ receptor II (TGFβRII) inhibitor is a peptide comprising the amino acid sequence ECGLLPVGRPDRVWRLCK (SEQ ID NO:1) and variations thereof

3. The method of claim 1, wherein the hydrogel comprises a thermoresponsive N-isopropylacrylamide (NIPAAm) hydrogel.

4. The method of claim 3, wherein the NIPAAm hydrogel comprises from about 2 wt % to about 20 wt % of NIPAAm.

5. The method of claim 4, wherein the NIPAAm hydrogel comprises from about 6 wt % to about 10 wt % of NIPAAm.

6. The method of claim 5, wherein the NIPAAm hydrogel comprises about 6 wt % of NIPAAm.

7. The method of claim 3, wherein the NIPAAm hydrogel comprises an injectable liquid phase at 4° C. and a gel phase at 37° C.

8. The method of claim 1, wherein the therapeutically effective dosage of the TGFβ receptor II (TGFβRII) inhibitor ranges from about 1 μg/mL to about 100 μg/mL.

9. The method of claim 8, wherein the therapeutically effective dosage of the TGFβ receptor II (TGFβRII) inhibitor ranges from about 1 μg/mL to about 10 μg/mL.

10. The method of claim 1, wherein the hydrogel is configured to degrade in the presence of reactive oxygen species (ROS).

11. The method of claim 1, wherein the hydrogel is synthesized by free radical polymerization using N-isopropylacrylamide (NIPAAm), 2-hydroxyethyl methacrylate (HEMA), and 4-(hydroxymethyl)-phenylboronic acid pinacol ester (HPPE).
